(19) United States
(12) Patent Application Publication
(10) Pub. No.: US 2006/0183226 A1
(43) Pub. Date: Aug. 17, 2006

(54) Methods for retrotransposing long interspersed elements (lines)

(76) Inventors: Haruhiko Fujiwara, Tokyo (JP); Hidekazu Takahashi, Baltimore, MD (US); Mamoru Hasegawa, Ibaraki (JP)

Correspondence Address: CLARK & ELBING LLP, 101 FEDERAL STREET, BOSTON, MA, 02110, US

(21) Appl. No.: 10/503,199

(22) PCT Filed: Nov. 26, 2002
(86) PCT No.: PCT/JP02/12317

§ 371 Date: Apr. 25, 2005

(30) Foreign Application Priority Data
Jan. 31, 2002 (JP) 2002-24226

(51) Int. Cl. C12N 15/86; C12N 15/74

(52) U.S. Cl. 435/456; 435/473

(57) ABSTRACT

The present invention provides methods for retrotransposing LINEs. The present invention relates to methods for transcribing RNAs comprising LINE 3′UTR fragments in cells, and retrotransposing these RNAs by using viral vectors to provide their LINE ORF proteins in trans. This invention also relates to methods for altering LINE retrotransposition target sites by replacing a LINE endonuclease domain with an endonuclease domain of another LINE. The methods of LINE retrotransposition of the present invention are useful for novel gene delivery.

### TECHNICAL FIELD

The present invention relates to methods for retrotransposing long interspersed elements (LINEs). The methods of the present invention are useful for target-specific introduction of nucleic acids into chromosomes.

### BACKGROUND ART

The recent progress of genome projects has revealed the existence of an abundance of transposable elements in higher eukaryotic genomes. Approximately 45% of the human genome is comprised of transposable elements (Lander, E. S. et al. (2001) Nature, 409, 860-921), and DNA transposons account for only 3% of these. The majority of transposable elements are retrotransposable elements, which are considered to transpose via RNA. Of these, the largest group is long interspersed elements (LINES) which make up 21% of the genome (Weiner, A. M. et al. (1986) Annu. Rev. Biochem., 55, 631-661; Smit, A. F. (1999) Curr. Opin. Genet. Dev., 6, 657-663) LINEs are a major class of retrotransposable elements. They transpose, via RNA intermediates, using self-encoding reverse transcriptase (RT) activity. LINEs shape mammalian genomes through de novo disease formation, exon shuffling, and mobilization of short interspersed elements (SINEs) and processed pseudogenes (Kazazian, H. H. et al. (1988) Nature, 332, 164-166; Moran, J. V. et al. (1999) Science, 283, 1530-1534; Esnault, C. et al. (2000) Nat. Genet., 24, 363-367). LINEs are also called non-LTR retrotransposons. Compared to LTR-retrotransposons and retroviruses, which use long terminal repeats (LTRs) that function as cis-elements essential for reverse transcription, the transposition mechanisms used by LINEs are relatively unknown (Boeke, J. D. and Stoye, J. P. (1997) Retrotransposons, endogenous retroviruses, and the evolution of retroelements. In Coffin, J. M., Hughes, S. H. and Varmus, H. E. (eds), Retroviruses. Cold Spring Harbor Laboratory Press, Cold Spring Harbor, N.Y., pp. 343-435).

LINEs can be classified into two subtypes (Malik, H. S. et al. (1999) Mol. Biol. Evol., 6, 793-805). One subtype is characterized by the existence of a restriction enzyme-like endonuclease domain to the 3′ side of the RT domain, and in most cases this type of LINE comprises a single open reading frame (ORF). The endonucleases encoded by this group show similarities with several motifs of amino acid residues observed in various prokaryote restriction enzymes (Yang, J. et al., 1999, Proc. Natl. Acad. Sci. USA 96: 7847-7852). The evolutionary origin of this group is ancient, and retrotransposition is directed to specific target sequences in all cases. In vitro biochemical analysis of one such element, R2, led to the current model for non-LTR retrotransposition. The protein encoded by the R2 ORF (proteins encoded by ORFs are also called “ORF proteins”) makes a specific nick on a 28S rDNA target site, and this nick is used to start the reverse transcription of its own RNA (Luan, D. D. et al. (1993) Cell, 72, 595-605). This mechanism is called target-primed reverse transcription (TPRT). However, little is known about the subsequent steps comprising synthesis of the second strand, and it is uncertain as to whether TPRT is widely utilized by other LINEs.

The other type of LINE is characterized by the existence of an apurinic/apyrimidinic-like endonuclease (APE) domain to the 5′ side of the RT domain, and comprises two ORFs in most cases. This group shows a broad distribution among eukaryotes, and comprises human L1, Drosophila factor I, and silk worm R1 (Hattori, M. et al. (1986) Nature, 321, 625-628; Fawcett, D. H. et al. (1986) Cell, 47, 1007-1015; Xiong, Y. and Eickbush, T. H. (1988) Mol. Cell. Biol., 8, 114-123). Two ORF proteins encoded by this type of LINE are poorly characterized. The ORF1 protein has been shown to form a cytoplasmic multimeric ribonucleoprotein complex (Hohjoh, H. and Singer, M. F. (1996) EMBO J., 15, 630-639; Dawson, A. et al. (1997) EMBO J., 16, 4448-4455; Pont-Kingdon, G. et al. (1997) Nucl. Acids Res., 5, 3088-3094), and to comprise nucleic acid chaperone activity (Martin, S. L. and Bushman, F. D. (2001) Mol. Cell. Biol., 21, 467-475). The second ORF encodes a protein comprising an N-terminal APE domain (Feng, Q. et al. (1996) Cell, 87, 905-916), a central RT domain (Mathias, S. L. et al. (1991) Science, 254, 1808-1810), and a C-terminal cysteine-histidine motif. An in vivo retrotransposition assay using a drug resistance marker was developed for human L1 to identify several ORF amino acid residues important for retrotransposition (Moran, J. V. et al. (1996) Cell, 87, 917-927). However, since L1 lacks insertion site specificity, further analysis of the retrotransposition mechanism and development of its application has been difficult.

### DISCLOSURE OF THE INVENTION

The present invention relates to methods for retrotransposition. Furthermore, the present invention provides methods for regulating target specificity during retrotransposition. This invention also provides novel vectors used for retrotransposition. The methods of the present invention are useful for gene delivery, for example, in gene therapy.

The present inventors used genetic engineering to study retrotransposable elements in order to develop novel gene delivery vectors able to integrate nucleic acids into cell chromosomes. TRAS and SART families have structures typical of the latter subtype of LINEs, described above, and comprise an APE domain at the 5′ side of their RT domain (Okazaki, S. et al. (1995) Mol. Cell. Biol., 15, 4545-4552; Takahashi, H. et al. (1997) Nucl. Acids Res., 25, 1578-1584). These families are highly transcribed in many tissues, and this transcription is driven by an internal promoter that is itself transcribed (Takahashi, H. and Fujiwara, H. (1999) Nucl. Acids Res., 27, 2015-2021). This type of LINE is 6 to 8 kb in length with two overlapping ORFs and a 3′ poly(A) tail. The amino acid sequence identity of the RT domains of TRAS1 (GenBank Ac. No. D38414) and SART1 (GenBank Ac. No. D85594) is a relatively low 29.3%. Although their gene organization is similar to that of human L1, TRAS1 and SART1 are unique in that they exist at specific nucleotide positions of the telomeric repeats, (TTAGG)ₙ, of silkworm Bombyx mori (Okazaki, S. et al. (1993) Mol. Cell. Biol., 13, 1424-1432; Sasaki, T. and Fujiwara, H. (2000) Eur. J. Biochem., 267, 3025-3031). Therefore, the TRAS and SART families can be good model systems for analyzing the retrotransposition of the latter subtype of LINEs.

The present inventors used SART1 and TRAS1 to develop a novel system that can be used to analyze in vivo LINE retrotransposition. The present inventors used the Autographa californica nuclear polyhedrosis virus (AcNPV) vector to express the B. mori SART1 element, under the control of the polyhedrin promoter comprised in this vector, in Spodoptera frugiperda cells (Sf9). Since S. frugiperda, like B. mori, belongs to the order Lepidoptera, and comprises (TTAGG) n repeats at telomeres (Maeshima, K. et al. (2001) EMBO J., 20, 3218-3228), retrotransposition was expected to occur in the host cell (Sf9) chromosomal telomeric repeats. Using this heterologous expression system, the present inventors demonstrated by an assay using polymerase chain reaction (PCR) that SART1 actually transposes into the telomeric repeats of the host chromosomes. The transposition site is in the same place as the specific nucleotide position of this element in the B. mori genome, and confirmatory retrotransposition by complete reverse transcription of the entire RNA transcription unit was observed. The retrotransposition required conserved domains in both of the two ORFs, which comprise the ORF1 cysteine-histidine motifs. In the present invention, RNAs were successfully retrotransposed by providing, in trans, proteins necessary for their transposition (i.e., these proteins are expressed from RNAs other than those being transposed). Recognition of the 3′ untranslated region (UTR) sequence is crucial for retrotransposition, and is known to result in retrotransposition by effective trans-complementation. The present inventors also found that in chimeric elements where the SART1 endonuclease domain is exchanged with that of TRAS1, the insertion specificity of retrotransposition is transferred to that of TRAS1. Therefore, the primary determinant of in vivo target selection was proved to be the endonuclease domain. Based on these findings, it is possible to impart LINEs with target site specificity, and in addition, to develop novel retrotransposition vectors that can introduce genes by trans-complementation. Modified LINEs, in which the proteins necessary for transposition are provided in trans, deliver only the genes of interest in trans to specific genomic locations. They are very useful as gene therapy vectors that do not deliver genes encoding the retrotransposon ORF proteins.

In the 21st century, gene therapy is expected to provide a means for treating genetic diseases. This requires stable human expression vectors. Currently, most gene delivery vectors are derived from retroviruses. These vectors are problematic in that they integrate randomly into genomes, and may disrupt essential genes. Therefore, it is important to develop gene delivery vectors that can be inserted into specific genome locations. To accomplish this objective, mobile group II introns have been engineered to facilitate insertion into specific sequences (Guo, H. et al. (2000) Science, 289, 452-457). However, since these introns are derived from bacteria, there is doubt as to whether they can be successfully expressed and retrotransposed into the genome in the case of living humans. In contrast, LINEs can be stably maintained in animal genomes. Therefore, LINEs are suitable candidates for mammalian transformation vectors. In fact, human L1 can retrotranspose into mouse cells (Moran, J. V. et al. (1996) Cell, 87, 917-927). Based on the results of chimeric SART1/TRAS1, the present inventors exchanged the APE domain with the APE domain of another site-specific LINE, showing that LINEs can be engineered to have target site specificity. Furthermore, since LINEs were shown to retrotranspose in trans, this system is advantageous in that ORFs can be separated from the sequences being retrotransposed. Such modified LINEs can be developed into harmless gene delivery vectors, which deliver only the genes of interest to a specific genomic site, and do not deliver the retrotransposons themselves. Thus it is thought that harmful retrotransposition into essential genes can be avoided, and stable protein expression can be achieved. An example of such a safe genomic location is the subtelomeric region. Using the endonuclease domain of LINEs that comprise specificity in a telomeric repeat allows the introduction of foreign genes into the subtelomeric region of chromosomes.

The present invention relates to methods for retrotransposing LINEs as well as vectors and such used for retrotransposition, and more specifically relates to:

(1) a method for retrotransposing an RNA, wherein the method comprises the steps of

(i) transcribing an RNA in a cell, wherein the RNA comprises a 3′UTR fragment of a LINE, and

(ii) expressing an ORF protein of the LINE, from somewhere other than the RNA;

(2) the method of (1), wherein the LINE is an APE domain-comprising LINE;

(3) the method of (1), wherein the LINE is a site-specific LINE;

(4) a method for retrotransposing an RNA, wherein the method comprises the steps of

(i) transcribing an RNA in a cell, wherein the RNA comprises a 3′UTR fragment of an APE domain-comprising site-specific LINE, and

(ii) expressing an ORF protein of the LINE in the cell;

(5) a method for retrotransposing an RNA, wherein the method comprises the steps of

(i) transcribing an RNA in a cell, wherein the RNA comprises a 3′UTR fragment of a LINE, and

(ii) expressing an ORF protein of the LINE in the cell, wherein the endonuclease domain of the ORF protein has been replaced with an endonuclease domain of another LINE;

(6) the method of (5), wherein the other LINE is an APE domain-comprising LINE;

(7) the method of (5), wherein the other LINE is a site-specific LINE;

(8) the method of any one of (3), (4), and (7), wherein the site-specific LINE is a telomeric repeat-specific LINE;

(9) the method of (8), wherein the telomeric repeat-specific LINE is a member of TRAS family or SART family;

(10) the method of any one of (1) to (9), wherein the ORF protein and/or the RNA is expressed from a viral vector;

(11) a retrotransposition vector encoding an RNA comprising a 3′UTR fragment of a LINE, wherein the vector does not express an ORF protein encoded by the LINE;

(12) a vector encoding an ORF protein encoded by a LINE, wherein the endonuclease domain of the protein has been replaced with an endonuclease domain of an ORF protein encoded by a site-specific LINE;

(13) the vector of (11) or (12), wherein the vector is a viral vector;

(14) the viral vector of (13), wherein the virus does not integrate into chromosomes;

(15) the viral vector of (14), wherein the virus that does not integrate into chromosomes is a baculovirus;

(16) a kit for gene delivery mediated by retrotransposition of an RNA, wherein the kit comprises

(i) a vector expressing an ORF protein encoded by a LINE, and

(ii) a vector that encodes an RNA comprising a 3′UTR fragment of the LINE, and which does not express the ORF protein;

(17) the kit of (16), wherein the ORF protein comprises an endonuclease domain of an ORF protein encoded by a site-specific LINE; and,

(18) the kit of (17), wherein the vector is a viral vector.

In the present invention, LINEs refer to DNAs that exist in eukaryote chromosomes, or their transcription products. These LINEs are long retrotransposable elements that do not comprise LTRs (long terminal repeats). The length of a natural LINE is normally 3 kb to 15 kb or so, and is preferably 4 kb to 10 kb or so. Typical LINEs encode, within themselves, ORFs that comprise an RT-like domain. However, LINEs that lack a complete ORF also exist (Malik H. S. et al., 1999, Mol. Biol. Evol. 16: 793-805). LINEs are also called non-LTR retroposons. Normally, as described above, a LINE ORF encodes a protein comprising an amino acid sequence homologous to a reverse transcriptase (RT), and often comprises poly(A) at its terminus. Examples of typical known LINEs are the elements described in Malik H. S. et al., 1999, Mol. Biol. Evol. 16: 793-805, and Xiong, Y. and Eickbush, T. H., 1988, Mol. Biol. Evol. 5:675-690. The amino acid sequences encoded by the ORFs maintained by LINEs share commonalities, and LINEs can be identified based on such characteristics. Phylogenetic analysis based on the amino acid sequences of the RT domains show that LINEs form a single group.

The present invention provides methods for retrotransposing RNAs that comprise a LINE 3′UTR fragment, by expressing these RNAs and LINE ORF proteins from separate vectors. The use of this kind of retrotransposition by trans-complementation enables-separation of the gene transfer vector and the vector that supplies the proteins required for transfer. Desired genes can be incorporated into gene transfer vectors, and by introducing such gene transfer vectors into target cells along with a vector that expresses a LINE ORF protein necessary for the transposition, the transcription products from the gene transfer vector are integrated into the chromosome. By designing ORF protein expression vectors that do not comprise the LINE 3′UTRs comprised in the gene transfer vectors, the transcription product of the ORF protein expression vector will not be integrated into the chromosome of the target cell. At the same time, by designing gene transfer vectors so as not to express ORF proteins, there is no danger of repeated transposition, even if a vector integrated once by retrotransposition is transcribed. This is because the ORF proteins necessary for transposition will not expressed. Therefore, gene transfer vectors encoding RNAs that comprise LINE 3′ UTR fragments can be prepared as vectors incapable of self-transposition, which lack the ability to retrotranspose on their own.

A “LINE 3′UTR fragment” of the present invention refers to the entire sequence of the 3′-side untranslated region (UTR) in a strand of LINE to be transcribed (sense strand), or a portion thereof. Where a LINE comprises a 3′-end poly(A) tail, 3′UTR fragments of that LINE preferably encompass a poly(A) sequence. When transcribing RNAs comprising poly(A) sequences, the length of the poly(A) sequence can be, for example, two to 100 nucleotides, preferably five to 60 nucleotides, and more preferably ten to 40 nucleotides (for example, approximately 20 nucleotides). The length of the 3′UTR fragment upstream of the poly(A) tail can be adjusted appropriately, as long as it shows retrotransposition activity. For efficient retrotransposition, it is preferable to comprise as long a region as possible. Specifically, the length of the 3′UTR fragment is preferably 20 nucleotides or more, more preferably 50 nucleotides or more, more preferably 100 nucleotides or more, more preferably 200 nucleotides or more, more preferably 250 nucleotides or more, and even more preferably 300 nucleotides or more. The LINE 3′UTR fragment necessary for retrotransposition activity is usually 3000 nucleotides or less, for example, 2000, 1000, 800 nucleotides or less. For example, a fragment comprising about 70% of the central portion of 3′UTR may be used suitably.

The LINE 3′UTR fragment can also be obtained from a LINE that is not full-length. LINEs in the genome often show 5′ deletions, but by isolating the 3′-end of such non-full-length LINEs, a retrotransposition vector can be constructed (Sassaman, D. M. et al. (1997) Nat. Genet., 16, 37-43; Ohshima, K. et al. (1996) Mol. Cell. Biol., 16, 3756-3764; Luan, D. D. and Eickbush, T. H. (1995) Mol. Cell. Biol., 15, 3882-3891; Jurka, J. (1997) Proc. Natl. Acad. Sci. USA, 94, 1872-1877).

The 3′UTR sequences may have one or more nucleotide deletions and/or insertions. For example, a sequence comprising the full-length sequence of a LINE 3′UTR can be preferably used as a LINE 3′UTR fragment of the present invention. An example of such a sequence is a nucleotide sequence from the nucleotide immediately after the ORF2 stop codon to the nucleotide at the 3′-end (or, in an element comprising poly A, to the nucleotide immediately before the poly A) Furthermore, in the present invention, the RNAs comprising 3′UTR fragments can also comprise LINE ORFs or portions thereof, in addition to the 3′UTRs. RNAs comprising ORFs or portions thereof can be made so as not to express an ORF2 protein or portion thereof. This can be achieved by deleting the initiation codon of that ORF, or by introducing a stop codon or frame shift mutation. Furthermore, RNAs comprising LINE 3′UTR fragments can comprise full-length LINE RNAs. For example, RNAs that do not express functional proteins, due to mutations introduced to the ORFs comprised in the full-length LINE sequence, can also be retrotransposed according to the present invention.

In the case of the SART1 3′UTR (SEQ ID NO: 52), of the 461 nucleotides, the 70 nucleotides from the 5′-end and 168 nucleotides from the 3′-end of 3′UTR, are not necessary for retrotransposition activity. Retrotransposition activity is only indicated by the 71st to 293rd nucleotides from the 5′-end. Therefore, a polynucleotide comprising the nucleotide sequence of position 71 to 293 from the 5′-end of 3′UTR (the nucleotide sequence of the position 71 to 293 of SEQ ID NO: 52) can be used as the 3′UTR fragment for retrotransposition. This suggests that the sequence required for retrotransposition is comprised within this sequence of approximately 200 nucleotides in the 3′UTR. However, the retrotransposition efficiency of RNAs comprising a short 3′UTR fragment is lower than that of RNAs comprising a long 3′UTR fragment or a full-length 3′UTR. When the poly A downstream of the 3′UTR is deleted, retrotransposition efficiency is decreased. Therefore, it is preferable that the length of LINE 3′UTR is as long as possible; for example, 250 nucleotides or more, preferably 300 nucleotides or more, more preferably 350 nucleotides or more, and even more preferably 400 nucleotides or more.

RNAs encoding LINE 3′UTR fragments and ORF proteins can be expressed in cells using a desired vector system. In a preferred embodiment, a viral vector is used. It is thought that the use of viral vectors to overexpress RNAs comprising LINE 3′UTR and/or ORF proteins may enable efficient trans-complementation of LINEs with a cis preference (Boeke, J. D. (1997) Nat. Genet., 16, 6-7; Wei, W. et al. (2001) Mol. Cell. Biol., 21, 1429-143; Okada, N. et al. (1997) Gene, 205, 229-243). Viral vectors that do not integrate into chromosomes are especially preferred as the viral vectors.

“LINE ORF proteins” refer to proteins encoded by ORFs carried by LINEs. ORF proteins may be natural LINE ORF proteins, and as long as the RNA comprising the LINE 3′UTR fragment is retrotransposed, may also be other LINE ORF proteins, or chimeric proteins with other LINE ORF proteins. For LINEs comprising two ORFs, “LINE ORF proteins” refer to proteins encoded by both the first ORF (ORF1) and the second ORF (OFR2). When LINES comprise multiple ORFs, ORF proteins may be derived from a different LINE for every ORF, however, they are preferably derived from the same LINE, except for the EN domain. In addition to forming chimeras with other LINE ORF proteins, ORF proteins may, for example, comprise mutations in their amino acid sequences, as long as retrotransposition activity exists.

Methods for artificially introducing mutations to amino acids include site-specific mutagenesis methods such as the Kunkel method (Kunkel, T. A., 1985, Proc. Natl. Acad. Sci. USA 82, 488; Kunkel, T. A. et al., 1987, Methods Enzymol. 154, 367), Gapped duplex method (Kramer, W. et al., 1984, Nucleic Acids Res. 12, 9441; Kramer, W. and Frits, H. J., 1987, Methods Enzymol. 154, 350), Eckstein method (Sayers, J. R. et al., 1992, Biotechniques, 13, 592), AlteredSite method (Lesley S. A. & Bohnsack, R. N., 1994, Promega Notes Magazine, 46, 6-10), Ito method (Ito, W. et al., 1991, Gene, 102, 67), PCR method (Cormack, B., in “Current Protocols in Molecular Biology” (Ausubel, F. M. et al., eds.), 8.5.1-8.5.9, 1987), or oligonucleotide ligation method (Uhlmann, E., 1988, Gene, 71, 29-40; Moore, D. D., 1987, in “Current Protocols in Molecular Biology” (Ausubel, F. M. et al., eds.), 8.2.8-8.2.13, 1987). Furthermore, nucleic acids encoding mutant proteins can be produced by introducing random mutations using the deletion method (Ausuber, F. M. et al., eds. in “Current Protocols in Molecular Biology”, 1.02-5.10.2, 1987; Sambrook, J. et al., in “Molecular Cloning A Laboratory Manual”, 2nd ed., 5.1-6.62, 1987), linker insertion method (Ausuber, F. M. et al., eds. in “Current Protocols in Molecular Biology”, 1.02-5.10.2, 1987; Sambrook, J. et al., in “Molecular Cloning A Laboratory Manual”, 2nd ed., 5.1-6.62, 1987), chemical mutagenesis (Myers, R. M., in “Current Protocols in Molecular Biology” (Ausubel, F. M. et al., eds.), 8.3.1-8.3.6, 1987), degenerate oligonucleotide method (Hill, D. E. et al., Methods Enzymol., 155, 558-568, 1987; Hill, D. E., in “Current Protocols in Molecular Biology” (Ausubel, F. M. et al., eds.), 8.2.1-8.2.7, 1987), linker scanning method (Greene, J. M. et al., Mol. Cell. Biol. 7, 3646-3655, 1987), or such. Mutations of amino acids may also occur in nature. Proteins which comprise amino acid mutations in the ORF proteins of wild-type LINEs and comprise retrotransposition activity can be used in the present invention, regardless of whether they are artificial or naturally occurring. Retrotransposition activity can be measured by PCR assay and so on, as described in the Examples.

The number of amino acids that are mutated in such mutants is not limited, but when artificially mutating an amino acid sequence, the mutated amino acids are normally 10% or less, preferably 5% or less, more preferably 3% or less, and most preferably 1% or less of all amino acids encoded by the ORF. More specifically, the number of mutated amino acids is normally 100 amino acids or less, preferably 80 amino acids or less, more preferably 60 amino acids or less, and even more preferably 30 amino acids or less (for example, ten amino acids). However, when amino acids are added to an ORF terminal (the N-terminal or C-terminal), the number is not particularly limited. Amino acids can be substituted, for example, with amino acids in corresponding positions in other LINE ORFs.

Furthermore, when artificially substituting amino acids, it is thought that the activity of the original protein is more easily conserved if amino acids whose side chains have similar chemical properties are substituted. Such conservative amino acid substitution is well known to those skilled in the art. This kind of amino acid group includes basic amino acids (for example, lysine, arginine, and histidine), acidic amino acids (for example, aspartic acid and glutamic acid), uncharged polar amino acids (for example, glycine, asparagine, glutamine, serine, threonine, tyrosine, and cysteine), non-polar amino acids (for example, alanine, valine, leucine, isoleucine, proline, phenylalanine, methionine, and tryptophan), β-branched amino acids (for example, threonine, valine, and isoleucine), and aromatic amino acids (for example, tyrosine, phenylalanine, tryptophane, and histidine).

LINE ORF proteins comprise a number of conserved motifs, and each of the motifs is characterized by amino acids conserved at specific sites. In LINE ORF proteins, it is preferable that these conserved amino acids are maintained as they are, particularly in natural LINE ORF proteins, or that they are substituted with amino acids having similar properties, as described above. Motifs conserved in LINE ORF proteins include amino acids conserved in the cysteine-histidine motif (otherwise called zinc finger motif or CCHC motif), endonuclease domain, and reverse transcriptase (RT) domain. When one or more cysteine-histidine motifs are present in each of a plurality of ORFs comprised by a LINE, it is preferable that all of these cysteine-histidine motifs are maintained. The conserved motifs and conserved amino acid residues of LINE ORF proteins are well known to those skilled in the art (Malik H. S. et al., 1999, Mol. Biol. Evol. 16: 793-805; Xiong, Y. and Eickbush, T. H., 1988, Mol. Biol. Evol. 5:675-690).

LINEs used in the retrotransposition methods of the present invention are preferably LINEs comprising an Exo_endo_phos domain (Pfam Accession number PF03372). More preferably, they are LINEs comprising an APE domain. An “APE domain” refers to an apurine/apyrimidine-like endonuclease domain, and LINEs possessing this domain show broad distribution amongeukaryotes, and form a major group among LINEs. As described above, LINEs are classified into those comprising the APE domain, and those that do not. The ORFs in each group can be found to have common structural and amino acid sequence characteristics. APE domain-comprising LINEs include various LINEs such as mammlian L1, Drosophila factor I, and insect R1 (Hattori, M. et al. (1986) Nature, 321, 625-628; Fawcett, D. H. et al. (1986) Cell, 47, 1007-1015; Xiong, Y. and Eickbush, T. H. (1988) Mol. Cell. Biol., 8, 114-123). Furthermore, the SART family and TRAS family, discovered in the telomeric repeat of insects, are also typical APE domain-comprising LINEs. Most of these LINEs comprise two ORFs (ORF1 and ORF2), and the APE domain is positioned near the N-terminal of ORF2. The open reading frames of ORF1 and ORF2 often overlap. APE domains can be identified by conserved amino acid residues. Amino acid residues characteristic of the APE domain have already been identified (Cost, G. J., and J. D. Boeke, 1998, Biochemistry 37:18081-18093; Feng, Q. et al., 1996, Cell 87:905-916; Christensen, S. et al., 2000, Mol. Cell. Biol. 20: 1219-1226; Feng, Q. et al., 1998, Proc. Natl. Acad. Sci. USA 95:2033-2088; Malik, H. S. et al., 1999, Mol. Biol. Evol. 16: 793-805; MartoAn, F. et al., 1995, J. Mol. Biol. 247:49-59; Freeland, T. M. et al., 1996, Nucleic Acids Res. 24:1950-1953). For example, identification of seven domains characteristic of the APE domain can determine the presence of an APE domain (McClure, M. A. et al. (2002) Virology 296: 147-158, FIG. 4).

The exo_endo_phos domain (PF03372) can be identified by a search based on the hidden Markov model (HMM) using a Protein families database of alignments and HMMs (Pfam) program (E. L. L. Sonnhammer, et al., 1997, Proteins 28:405-420; Bateman, A. et al. (2002) Nucleic Acids Res. 30(1): 276-280; Bateman, A. et al. (2000) Nucleic Acids Res. 28: 263-266; Bateman, A. et al. (1999) Nucleic Acids Res. 27: 260-262; Sonnhammer, E. L. L. (1998) Nucleic Acids Res. 26: 320-322; Sonnhammer, E. L. L. (1997) Proteins 28:405-420). Pfam 7.1 program and such may be used for Pfam (A. Bateman et al., 2002, Nucleic Acids Res. 30: 276-280). When the score (bit value) of the Exo_endo_phos domain with respect to a query sequence is 11.0 or more in ls mode (Pfam_ls) (HMM construction: hmmbuild -F HMM_ls.ann SEED.ann; hmmcalibrate --seed 0 HMM_ls.ann), or 19.6 or more in fsmode (Pfam_fs) (HMM construction: hmmbuild -f -F HMM_fs.ann SEED.ann; hmmcalibrate --seed 0 HMM_fs.ann), this sequence is identified as the sequence of the Exo_endo_phos domain. Preferably, the bit value of Pfam_ls is 11.6 or more and/or the bit value of Pfam_fs is 19.9 or more. More preferably, the bit value in is mode is 15 or more, more preferably 20 or more, even more preferably 30 or more, and most preferably 40 or more. Alternatively, the bit value in fs mode is 25 or more, preferably 30 or more, more preferably 35 or more, and most preferably 40 or more. The Expectation (E) value when homeoboxes are detected in this manner by Pfam is usually less than 1×10⁻³, preferably less than 1×10⁻⁵, more preferably less than 1×10⁻⁷, even more preferably less than 1×10⁻⁹, and yet even more preferably less than 1×10⁻¹¹. Pfam searches can be performed using a server in a website (Sanger Institute (UK), St. Louis (USA), Karolinska Institutet (Sweden), or Institut National de la Recherche Agronomique (France)), or a Pfam database can be downloaded from an FTP site to perform searches locally.

The above-described LINEs of the present invention are preferably site-specific LINEs. Site-specific LINEs refer to LINEs found at specific sites in host genomic DNAs. LINEs are categorized into a group that inserts into a variable DNA sequence, and a group that inserts into a specific nucleotide sequence. The former LINEs, which are randomly inserted, are represented by mammalian L1, and although some preference exists for their insertion site, the nucleotide sequence in which insertion occurs is hardly conserved at all. In contrast, site-specific LINEs are inserted into specific nucleotide sequences, and usually, the nucleotide position where insertion takes place is exactly the same. For example, Tx1 of Xenopus laevis is inserted into another transposon factor (Garrett, J. E. et al., 1989, Mol. Cell. Biol. 9:3018-3027). CRE1, SLACS, and CZAR are found in the splice leader exon of Trypanosoma (Aksoy, S. et al., 1990, Nucleic Acids Res. 18: 785-792; Gabriel, A. et al., 1990, Mol. Cell. Biol. 10: 615-624; Villanueva, M. S. et al., 1991, Mol. Cell. Biol. 11:6139-6148). R1 and R2 exist at specific positions of 28S rDNA in most insects (Eickbush, T. H. and Robins, B., 1985, EMBO J. 4: 2281-2285; Fujiwara, H. et al., 1984, Nucleic Acids Res. 12: 6861-6869; Jakubczak, J. L. et al., 1991, Proc. Natl. Acad. Sci. USA 88: 3295-3299). In two species of mosquitoes, RT1 and RT2 are inserted at the same positions, approximately 630 bp downstream of the R1 insertion site (Besansky, N. et al., 1992, Mol. Cell. Biol. 12: 5102-5110; Paskewitz, S. M. and Collins, F. H., 1989, Nucleic Acids Res. 17: 8125-8133). These LINEs are site-specific LINEs. Of these, R2, CRE1, and CZAR comprise one ORF, and encode a non-APE-type endonuclease near the C-terminal. This region is characterized by a common motif, Lys/Arg-Pro-Asp-x₁₂₋₁₉-Asp/Glu (PDD). On the other hand, L1, R1, Tx1L, and such comprise two ORFs, and carry an APE domain at the N-terminal of ORF2. The LINEs in the methods of the present invention are most preferably such APE domain-comprising site-specific LINEs. Examples of such LINEs include APE domain-comprising LINEs, especially those that are specifically inserted into the telomeric repeat of eukaryotes. The SART family and TRAS family are site-specific LINEs comprising an APE domain, and positioned at specific nucleotide positions in the telomeric repeat. The use of LINEs of the SART family and TRAS family is especially preferred in the present invention.

The present invention also provides retrotransposition systems for APE domain-comprising site-specific LINEs. Using the methods provided by the present invention, APE domain-comprising site-specific LINEs can be retrotransposed according to their target directionality. The present inventors used a site-specific LINE, SART1, to establish an in vivo retrotransposition system. In this system, the RNA comprising the 3′UTR fragment of a APE domain-comprising site-specific LINE, and the ORF proteins of this LINE, are expressed in cells that comprise the target DNA of this LINE. The ORF proteins expressed in the cells recognize the RNA comprising the LINE 3′UTR fragment, and site-specifically retrotranspose this RNA. In the retrotransposition methods of the present invention, the use of viral vectors to express RNAs and/or ORF proteins was found to be extremely preferable. The present invention provides, in particular, viral vectors encoding 3′UTR fragments of APE domain-comprising site-specific LINEs. These viral vectors enable efficient induction of retrotransposition. The present invention also relates to viral vectors that express the ORF proteins of APE domain-comprising site-specific LINEs. Viral vectors that do not integrate into chromosomes are expecially preferred as the viral vectors. Viral vectors that do not integrate into chromosomes comprise both DNA viral vectors and RNA viral vectors. Examples of particularly preferable viral vectors include DNA viral vectors that do not integrate into chromosomes, such as baculoviral vectors.

The present inventors developed methods for efficiently retrotransposing SART1 by inserting SART1, which targets telomeric repeats, into a viral vector, and infecting cells with this viral vector. Furthermore, using TRAS1, the present invention succeeded in retrotransposition that targets telomeric repeats. The present invention relates to methods for retrotransposing LINEs of the SART family and TRAS family to telomeric repeats by transfecting cells with vectors that express members of these families. Similarly, by inserting a desired APE domain-comprising site-specific LINE into a viral vector or such, and introducing this into cells comprising the target DNA, the LINE transcribed from the vector can be site-specifically retrotransposed. As shown in Example 4, by using a viral vector to express a full-length RNA of a LINE, or an RNA encoding a portion comprising a LINE ORF and a 3′UTR fragment, ORF proteins expressed from these RNAs can retrotranspose their own RNAs. Therefore, the present invention comprises vectors that encode 3′UTR fragments of APE domain-comprising site-specific LINEs, and express LINE ORF proteins. To construct such vectors, for example, a full length LINE, or a portion comprising a complete ORF and a 3′UTR fragment, is inserted into an expression vector such as a viral vector. In order to express foreign proteins from these RNAs that express LINE ORF proteins, for example, internal ribosomal entry sites (IRES), or incomplete splicing may be utilized. Alternatively, the ORF portion and the 3′UTR fragment can be expressed as separate transcription units from the same vector. Furthermore, the vectors that transcribe RNAs encoding 3′UTR fragments, and the vectors expressing ORF proteins can be separated in order to transpose site-specific LINEs by trans-complementation.

The present inventors have identified several families of APE-comprising site-specific LINEs in telomeric repeats (Okazaki, S. et al., 1995, Mol. Cell. Biol. 15: 4545-4552; Takahashi, H. et al., 1997, Nucleic Acids Res. 25: 1578-1584). The present inventors were the first to use these LINEs to successfully induce retrotransposition that targets telomeric repeats. The present invention enables RNAs to be retrotransposed into telomeric repeats using LINEs comprising site specificity to these telomeric repeats. Such LINE families include the TRAS family and SART family. The TRAS family and SART family are APE-comprising LINEs that are inserted in opposite directions into the telomeric repeat, (TTAGG)ₙ, of insect subtelomeric regions (Okazaki, S. et al., 1995, Mol. Cell. Biol. 15: 4545-4552; Takahashi, H. et al., 1997, Nucleic Acids Res. 25: 1578-1584). The TRAS family comprise a sense strand in the CA-rich strand of the telomeric repeat, and the SART family comprise a sense strand in the GT-rich strand. Each family has many members, and they comprise common structural characteristics. For example, members of the TRAS family include TRAS1, TRAS3, TRAS4, TRAS5, TRAS6, TRASY, TRASZ, TRASW, TRASDJ, TRASSC3, TRASSC4, and TRASSC9. SART1 and SART2 have been identified in the SART family. The amino acid sequences of ORF proteins encoded by members of the same family are highly homologous, and members of the TRAS family or SART family can thus be identified based on this homology (Kubo, Y. et al., 2001, Mol. Biol. Evol. 18(5): 848-57; WO01/88149). For example, the amino acid sequence homology of the region from the endonuclease domain to the RT domain is compared with any one of the identified members of the SART or TRAS families. If the amino acid sequence identity is significantly higher than that of a member of another family closely related to the SART or TRAS family (for example R1), this sequence can be determined to belong to the SART or TRAS family, respectively. For example, if the amino acid sequence from the endonuclease domain to the RT domain, or a similar region, comprises about 31% or more, more reliably about 33% or more, more preferably about 35% or more, and even more preferably about 37% or more (for example, about 40% or more), identity to any of the identified members of the TRAS family, this element is considered to be a member of the TRAS family. Members of the TRAS family comprise nucleotide sequence identity of about 45% or more, more reliably about 47% or more, more preferably about 50% or more, and even more preferably about 52% or more to any one of the identified members of the TRAS family in the coding region of this amino acid sequence. Members of the SART family can be similarly identified.

Amino acid or nucleotide sequence identity can be determined using a known computer program. For example, amino acid or nucleotide sequences can be aligned by an alignment program such as CLUSTAL W (Thompson, J. D. et al., 1994, Nucleic Acids Res. 22: 4673-80), and identity can be calculated by counting the matching amino acid residues or nucleotides. Gaps are treated in the same way as mismatches, and identity can be calculated as the ratio of matched nucleotides within the total number of nucleotides comprising the gaps. Alternatively, programs such as blastn or blastp can be used (Altschul, S. F. et al. (1990) J. Mol. Biol. 215: 403-410; Gish, W. & States, D. J. (1993) Nature Genet. 3: 266-272; Madden, T. L. et al. (1996) Meth. Enzymol. 266: 131-141; Altschul, S. F. et al. (1997) Nucleic Acids Res. 25: 3389-3402; Zhang, J. & Madden, T. L. (1997) Genome Res. 7:649-656). For example, in BLAST 2 SEQUENCES, which compares two amino acid sequences or nucleotide sequences by blastp or blastn, respectively (see Tatiana A. Tatusova, Thomas L. Madden (1999), “Blast 2 sequences—a new tool for comparing protein and nucleotide sequences”, FEMS Microbiol. Lett. 174: 247-250; the NCBI website for BLAST 2 SEQUENCES (http://www.ncbi.nlm.nih.gov/blast/bl2seq/bl2.html)), BLOSUM62 is used as the matrix for scoring when comparing amino acid sequences (Henikoff, Steven and Jorga G. Henikoff (1992) Amino acid substitution matrices from protein blocks. Proc. Natl. Acad. Sci. USA 89: 10915-19) (Open gap penalty: 11, extension gap penalty: 1). Identity values can be obtained as Identities (%) by searching without the use of FILTER (filtering of Low-complexity sequences).

Members of the SART family or TRAS family can also be identified by phylogenetic grouping. For example, members of the SART or TRAS family form groups with known SART or TRAS family, in which members of other families closely related to the SART or TRAS family (for example R1) are not comprised. Grouping can be performed by conventional methods based on the nucleotide sequences of DNAs or the amino acid sequences encoded thereby. For example, a phylogenetic tree is constructed based on the amino acid sequence from the endonuclease domain to the RT domain. Any desired hierarchical method comprising neighbor-joining method and maximum likelihood method can be used for the construction of the phylogenetic tree. The neighbor-joining method can be used as a preferable example (Saitou, N. and Nei, M., 1987, Mol. Biol. Evol. 4: 406-425). The reliability of the group can be evaluated by bootstrap probability. Preferably, the bootstrap probability that separates a certain family from the others is 50% or more, more preferably 80% or more, even more preferably 90% or more, and most preferably 95% or more (for example, 99.0% or more). Trials may be carried out 1000 times, for example.

Site-specific LINEs retrotransposed by the methods of the present invention can be detected by Southern blotting of host chromosomal DNA, or by in situ hybridization of chromosomes such as FISH. In particular, since retrotransposition of site-specific LINEs occurs at fixed insertion sequences, it can be simply assayed using polymerase chain reaction (PCR) (Sambrook, J et al., Molecular Cloning 2nd ed., 9.47-9.58, Cold Spring Harbor Lab. press, 1989; “The PCR Technique: DNA sequencing” (Eds. J. Ellingboe and U. Gyllensten), “BioTechniques Update Series”, Eaton Publishing, 1999; “The PCR Technique: DNA sequencing II” (Eds. U. Gyllensten and J. Ellingboe), “BioTechniques Update Series”, Eaton Publishing, 1999; “PCR Technology: principles and application for DNA amplification” Ed by H. A. Erlich, 1989, Stockton Press). More specifically, one primer is designed for the RNA portion that is transposed, and the other primer is designed for the sequence of the target site, and by performing a PCR amplification on the portion between these borders, the retrotransposed RNA alone can be specifically detected (see Examples). By combining the retrotransposition systems that use the above-mentioned viral vectors, highly effective systems that can analyze the retrotransposition of site-specific LINEs can be constructed.

In vivo retrotransposition of LINEs without site specificity has been previously indicated in several LINEs using plasmid vectors, based on splicing out of artificial introns (Jensen, S. and Heidmann, T. (1991) EMBO J., 10, 1927-1937; Pelisson, A. et al. (1991) Proc. Natl. Acad. Sci. USA, 88, 4907-4910; Evans, J. P. and Palmiter, R. D. (1991) Proc. Natl. Acad. Sci. USA, 88, 8792-8795; Kinsey, J. A. (1993) Proc. Natl. Acad. Sci. USA, 90, 9384-9387). Human Ll marker selection assays identified amino acid residues essential for retrotransposition (Moran, J. V. et al. (1996) Cell, 87, 917-927). However, since L1 and such do not have target sequence specificity, detailed analysis of the transposition mechanism is difficult. According to the present invention, the endonuclease domain of site-specific LINEs such as SART1 and TRAS1 can be utilized to develop a novel assay for detecting target-specific in vivo LINE transposition. In the assay using the system of the present invention, retrotransposition can be detected by PCR within two to three days, and the kinetics of retrotransposition can be analyzed in more detail. Accordingly, for example, when developing novel LINEs by exchanging the LINE ORF domain described later, testing and analysis that are more convenient than conventional L1 cultured cell assays, and directly linked to the retrotransposition reactions, can be performed. For example, the role of the domain in retrotransposition can be rapidly elucidated. Retrotransposition assay systems are useful in the analysis of LINE retrotransposition mechanisms, performance evaluation of gene transfer vectors, detection of retrotransposition in actual treatment, diagnosis, etc.

Furthermore, the present invention provides methods of exchanging the endonuclease domain of a LINE ORF protein with that of another LINE to alter the target site. The present inventors constructed a LINE in which the SART1 endonuclease domain was replaced with that of TRAS1, and performed retrotransposition by a method of the present invention. Surprisingly, this chimeric LINE showed the same target directivity as TRAS1. This result shows that the LINE endonuclease domain determines the target directivity of LINE in vivo. Therefore, by replacing the endonuclease domain of LINEs that are not target specific with the endonuclease domain of a site-specific LINE, a desired LINE can be exchanged with a site-specific LINE. On the other hand, the endonuclease domain of a site-specific LINE can be replaced with the endonuclease domain of LINE without site specificity to remove the target site specificity of that LINE. In this way, by exchanging LINE endonuclease domains, the targeting of LINE retrotransposition can be controlled according to the targeting of the endonuclease domain.

The range of LINE endonuclease domains can be identified based on amino acid sequence alignments (Kubo, Y. et al., 2001, Mol. Biol. Evol. 18(5): 848-57; WO01/88149). Amino acid sequence alignment can be performed by utilizing a computer program based on algorithms such as the above-mentioned BLAST (Karlin, S. and S. F. Altschul, 1990, Proc. Natl. Acad. Sci. USA 87: 2264-68; Karlin, S. and S. F. Altschul, 1993, Proc. Natl. Acad. Sci. USA 90: 5873-7), or CLUSTAL W (Thompson, J. D. et al., 1994, Nucleic Acids Res. 22: 4673-80).

More specifically, identification can be performed based on the description in “Malik, H. S. et al. (1999) Mol. Biol. Evol., 6, 793-805”. Alternatively, the range of the endonuclease domain can be specified, for example, by preparing an alignment comprising the consensus sequence of the Exo_endo_phos domain (PF03372) (SEQ ID NO: 51) together with appropriate gaps. The above-mentioned Pfam program can be utilized to prepare alignments. Amino acid sequences of the range specified by this alignment can be considered to be endonuclease domains. The N-terminal and C-terminal of a selected domain may be shorter or longer than both terminals of SEQ ID NO: 51. In the alignment with SEQ ID NO: 51, both terminals of an endonuclease domain may be different from those of SEQ ID NO: 51, for example, by seven amino acids or less, preferably six amino acids or less, and more preferably five, four, or three amino acids or less. A specific example uses the same range as that of the TRAS1 APE domain (PYRV . . . IRLQ), as indicated in FIG. 6.

The effect may be enhanced or made more reliable by exchanging regions other than ORF, in addition to exchange of the endonuclease domain. In vivo, genomic DNA is associated with many binding proteins in the form of chromatin. Considering this, and as proven with several LTR retrotransposons (Kirchner, J. et al. (1995) Science, 267, 1488-1491; Xie, W. et al. (2001) Mol. Cell. Biol., 19, 6606-6614) and suggested with human L1 (Cost, G. J. et al. (2001) Nucl. Acids Res., 29, 573-577), host chromatin protein interaction with other LINE ORF protein domains may be involved in target site selection. Therefore, by transplanting other domains in addition to the APE domain of site-specific LINE ORF proteins, there can be greater assurance of exchange of LINE target specificity (Feng, Q. et al. (1996) Cell, 87, 905-916; Feng, Q. et al. (1998) Proc. Natl. Acad. Sci. USA, 95, 2083-2088; Christensen, S. et al. (2000) Mol. Cell. Biol., 20, 1219-1226; Anzai, T. et al. (2001) Mol. Cell. Biol., 21, 100-108). For example, TRAS1 ORF2 encodes a region comprising weak homology with the Myb domain, found in many telomere-binding proteins at the center between the APE and RT domains (Kubo, Y. et al. (2001) Mol. Biol. Evol., 18, 848-357). Another domain such as this putative Myb domain may guarantee “telomere specificity” by recognizing the telosomes, and subsequent APE cleavage may determine the insertion site. Therefore, when exchanging endonuclease domains of APE-comprising LINEs, exchanging the Myb domain together with the APE domain may be preferable. Besides the Myb domain, for example the TRAS-specific region (TSR), which comprises twelve amino acids and is also conserved in the TRAS family (WO01/88149; Kubo, Y. et al., 2001, Mol. Biol. Evol. 18(5):848-57), may contribute to the precise recognition of telomeric repeats. Therefore, when exchanging APE domains, it is preferable that the downstream portion of APE is also exchanged. For example, it is preferably to exchange the region from the APE domain to just before the RT domain.

The SART and TRAS families can be retrotransposed into the telomeric repeats, (TTAGG), of insects, as well as into the telomeric repeats of other eukaryotes. Telomeric repeats are generally highly conserved in eukaryotes (“Telomeres” (Eds. E. H. Blackburn and C. W. Greiner) CSHL Press, 1995, Chapter 2 by E. Hendreson “Telomere DNA structure” pp 11-34; “The Telomere” by D. Kipling, Oxford Univ Press, 1995, Chapter 3 “Telomere structure” 31-69; Zakian, V. A. (1995) “Telomeres: beginning to understand the end” Science, 270, 1601-1607). Furthermore, the APE domain of TRAS1 cleaves not only the insect telomeric repeat, (TTAGG)ₙ, but also the (TTAGGG)ₙ telomeric repeat, which is conserved in vertebrates, including humans (WO01/88149). In the present invention, the endonuclease domain was shown to be the major determining factor in target selection for retrotransposition in cells. Thus, LINEs comprising APE domains of the SART and TRAS families may be retrotransposed in cells to the vertebrate-type telomeric repeats.

With regards to the vectors that encode RNAs comprising LINE 3′UTR fragments and constructed so as to not express ORF proteins encoded by these LINEs, RNAs transcribed from such vectors cause retrotranspoition by supplying the ORF protein in trans. However, since the ORF proteins are not expressed after transposition, transposition is not repeated. The present invention especially provides such retrotransposition vectors. Using the retrotransposition vectors of the present invention, desired nucleic acids can be integrated into the chromosomes of target cells. If the introduced nucleic acids do not need to be expressed after retrotransposition, a vector can be prepared whereby the nucleic acid sequence transcribes the RNA bound to the 5′ side of LINE 3′UTR fragment. Retrotransposition by such vectors is useful, for example, for integrating marker nucleic acid sequences into chromosomes, or in enhancer traps and such. In RNAs, sequences that function as promoters after retrotransposition can be comprised in the transcription products. Examples of preferable vectors are vectors comprising a “promoter; gene to be introduced or a cloning site for its insertion; LINE 3′UTR fragment”. More preferably, a poly(A) addition signal follows the LINE 3′UTR fragment. Promoters can be selected appropriately, but in the case of vectors comprising a single promoter, it is preferable to use an internal promoter, which itself is transcribed and has activity after retrotransposition (Takahashi, H. and Fujiwara, H. (1999) Nucl. Acids Res., 27, 2015-2021). Internal promoters have been identified in many LINEs, and LINES are generally thought to carry them. Furthermore, they are common to LTR-type transposons in insects and such (Archipova, I. R. et al., EMBO J. (1991) 10, 1169-1177), and genes relating to development (homeotic genes such as Antennapedia and Engrailed) (Takahashi, H. et al. (1997) Nucl. Acids Res., 25, 1578-1584; Takahashi, H. and Fujiwara, H. (1999) Nucl. Acids Res., 27, 2015-2021).

In addition, vectors with doubled promoters are also suitable. The present invention comprises such vectors comprising a double promoter structure. A specific example of the structure is a vector comprising “the first promoter; the second promoter; the gene to be introduced or a cloning site for its insertion; LINE 3′UTR fragment; poly(A) addition signal”. Products transcribed from the vector by the first promoter can express the inserted genes from the second promoter after retrotransposition. The second promoter can also be an internal promoter. Examples of such preferable vectors are vectors comprising a structure of “promoter; internal promoter; the gene to be introduced or a cloning site; LINE 3′UTR fragment; poly(A) addition signal”. Furthermore, sequences comprising a second promoter and a gene to be introduced can also be encoded by the antisense strand of the strand that is transcribed during transcription (comprising a LINE 3′UTR fragment in the sense direction) (see FIG. 7).

To prevent transcription during transcription from the vector, an appropriate regulatory sequence can be integrated before, after, or within the second promoter that is to function after retrotransposition. Such regulatory sequences include repressor sequences, introns, and recombinant signals such as loxP. Insertion of a foreign gene downstream of the second promoter enables the expression unit of the desired foreign gene to be retrotransposed. The foreign genes are not particularly limited, and any gene that whose expression in target cells is desired can be inserted. For example, a foreign gene of 2 kb or more can be inserted. In gene therapy, for example, a therapeutic gene is inserted.

The above-mentioned transcription product of a retrotransposition vector can be retrotranscribed by expressing a LINE ORF protein in cells, where the LINE ORF protein recognizes the LINE 3′UTR fragment comprised in the transcription product. LINE ORF proteins can be expressed by introducing vectors that express them into cells. Herein, replacement of the endonuclease domain of a LINE ORF protein with that of another LINE enables alteration of target specificity. In particular, replacing the endonuclease domain of a site-specific LINE enables an RNA to be specifically retrotransposed to that target site. The present invention provides vectors encoding LINE ORF proteins, where the endonuclease domain of an ORF protein encoded by a LINE has been replaced with that of an ORF protein encoded by a site-specific LINE. Replacement of the endonuclease domain may occur over the entire region or a portion of the endonuclease domain. When replacing a portion, the corresponding portions of two endonuclease domains are exchanged. The corresponding portions of two endonuclease domains can be identified as such by aligning both amino acid sequences. ORF proteins can be expressed by inserting a region comprising a LINE ORF downstream of a promoter, which is comprised in a known expression vector. In order to use trans-complementation to retrotranspose transcription products obtained from retrotransposition vectors, vectors expressing ORF proteins preferably lack LINE 3′UTR sequences. In this manner, its own transcription product is not recognized; only other RNA molecules comprising LINE 3′UTR fragments are recognized.

Furthermore, the present invention provides kits comprising vectors that express ORF proteins encoded by LINEs, and vectors that encode RNAs comprising 3′UTR fragments of those LINEs and that do not express the ORF proteins, wherein the kits are for gene delivery mediated by retrotransposition of the RNAs. Those ORF proteins in which the endonuclease domain has been replaced with the endonuclease domain of another LINE, as mentioned above, can be preferably used.

The above-mentioned retrotransposition vectors and LINE ORF protein expression vectors can be constructed using known vector systems, but are preferably constructed as viral vectors. By using viral vectors, vectors are introduced efficiently into host cells, and RNAs and ORF proteins can be expressed at high levels. Those viral vectors that do not integrate into chromosomes are especially preferable. By using this type of vector, components necessary for retrotransposition can be transiently expressed in target cells. Since these vectors will be removed from cells over time, they will not be unnecessarily expressed after retrotransposition is complete, and are therefore excellent vectors. Examples of viral vectors that do not integrate into chromosomes include adenovirus vectors (for example, pShuttle, Clontech), Sendai virus vectors, vaccinia virus vectors, Epstein-Barr virus vectors, baculovirus vectors, herpes virus vectors, and sindbis virus vectors (Soifer, H. et al., 2001, Hum. Gene Ther. 12: 1417-1428; Kay, M. et al., 2001, Nat. Med. 7: 33-40). By using vector that integrates into chromosomes, the integration site may be regulated. Examples of vectors that integrates into chromosomes include retrovirus vectors, lentivirus vectors, adeno-associated virus vectors, and foamy virus vectors. These viral vectors can be prepared by methods well known to those skilled in the art. Viral vectors can be purified, for example, by centrifugation, according to their types.

In order to express the vectors of the present invention in animals, in vivo or ex vivo, DNA vectors such as plasmids can be administered together with transfection reagents such as cationic lipids or liposomes. Naked DNAs or viral vectors can be directly administered. Examples of administration targets are humans and non-human mammals, and administration can be performed ex vivo or in vivo, to cells, tissues, organs, and such. Administration to a living body may be performed ex vivo or in vivo. In in vivo methods, the vector of the present invention is administered directly to a living body. In ex vivo methods, administration to cells outside a living body is followed by administration of those cells into a living body. In ex vivo methods, for example, cells producing a viral vector of the present invention may be administered. When administering locally to a target tissue, vectors or cells are administered to the target tissue via an injection needle, catheter, or such. Alternatively, vectors can be introduced to target tissues using carriers that can deliver vectors to specific tissues. Thus, the vectors of the present invention can be specifically retrotransposed to tumor cells and such.

The vectors of the present invention can be mixed with known carriers and vehicles to form composites. The vectors of the present invention can also be administered as pharmaceutical compositions that are formulated by conventional preparation methods. For example, they can be prepared as compositions by mixing with pharmaceutically acceptable carriers or vehicles, which specifically include sterilized water or physiological saline, salts, vegetable oil, stabilizers, preservatives, suspensions, and emulsifiers. Furthermore, the vectors of the present invention can be prepared as compositions for introducing nucleic acids into cells together with liposomes or cationic lipids.

When administered as pharmaceutical agents to a living body, the vectors of the present invention can generally be administered locally or systemically by methods well known to those skilled in the art, such as intraarterial injection, intravenous injection, subcutaneous injection, and intramuscular injection. Alternatively, they can be administered locally through a syringe, catheter, needle-less injector, or such. Dosage can vary depending on a patient's weight and age, the method of administration, and the symptoms, but one skilled in the art can appropriately select an appropriate dose. Administration can be performed once, or a number of times. Administration of the vectors of the present invention can be performed according to conventional gene therapy protocols.

### BRIEF DESCRIPTION OF THE DRAWINGS

FIG. 1 shows a PCR assay for in vivo SART1 retrotransposition. (A) is a schematic overview of the PCR assay. The hexagon represents SART1-expressing AcNPV, which was infected to Sf9 cells. As illustrated, SART1 is expected to retrotranspose into the telomeric repeats of the Sf9 chromosomes. Black arrows show primers used in PCR to detect the boundary between the transposed SART1 and the telomeric repeats. (B) is a detailed scheme of the assay. The Sf9 telomeric repeats (TTAGG/CCTAA)ₙ are shown in the middle. The schematic structure of SART1 expressed from AcNPV is shown at the top. The ORF1/ORF2/3′UTR is indicated as a gray box (not to scale) APE and RT denote the endonuclease domain and reverse transcriptase domain, respectively. Vertical lines represent cysteine-histidine motifs near the C-terminals of both ORFs. Note that ORF1 is fused in frame with the vector-derived GST-(His)₆ gene for future biochemical analysis. The black rectangle represents the polyhedrin promoter that drives transcription. Nucleotide positions are numbered with the transcription initiation site (A of TAAG) defined as +1. White arrows denote a pair of primers, +6276 and (CCTAA)₆, which were used in the experiment shown in FIG. 2 to amplify the boundary between SART1 3′ ends and the telomeric repeat. Thick black arrows indicate a pair of primers, +590 and (TTAGG) 6, which were used for the 5′ boundary amplification in the experiment shown in FIG. 3. The structure of TRAS1 expressed from AcNPV assayed in FIG. 6 is also shown at the bottom. RH denotes the RNase H domain. As the dotted arrows indicate, SART1 and TRAS1 are inserted between the TT and AGG nucleotides in the opposite orientation relative to the telomeric repeats. Note that correct insertion positions have a one-base uncertainty due to the repetitive nature of the poly(A) tail and the telomeric repeat, and that target site duplications have not been identified.

FIG. 2 shows the 3′ boundary analysis for retrotransposed SART1 elements. (A) shows a PCR amplification of the boundaries between the transposed SART1 3′ ends and the telomeric repeats. Sf9 cells were infected with AcNPV expressing wild-type SART1 or 2D699V, and the Sf9 genomic DNAs were extracted 7, 24, 48, and 72 hours post-infection (hpi). The purified DNAs were used as templates for PCR with a pair of primers, +6276 and (CCTAA) 6, described in FIG. 1B. The PCR products were subjected to 3% agarose gel electrophoresis and stained with ethidium bromide. A molecular size marker was run in the rightmost lane, and some of these base-pair sizes are indicated. (B) shows the nucleotide sequences of 29 clones from the 3′ boundary PCR products shown in lane 4 of panel (A). The number of each type (number of clones) is shown on the right. Nucleotide positions are indicated with the polyhedrin transcription initiation site defined as +1. The octanucleotide with homology to the telomeric repeat is underlined.

FIG. 3 shows the 5′ boundary analysis for retrotransposed SART1 elements. (A) shows the PCR amplification of the boundaries between the transposed SART1 5′ ends and the telomeric repeats. The AcNPV-infected Sf9 genomic DNAs were amplified with a pair of primers, +590 and (TTAGG) 6, shown in FIG. 1B. The PCR products were subjected to 3% agarose gel electrophoresis and stained with ethidium bromide. A molecular size marker was run in the rightmost lane, and some of these base-pair sizes are indicated. (B) shows the nucleotide sequences of 24 clones from the whole 5′ boundary PCR products in lane 4 of panel (A). The number of each type (number of clones) is shown on the right. Nucleotide positions are indicated with the polyhedrin transcription initiation site defined as +1. (C) shows SART1 retrotransposition with 5′ aberrations. The full-length 5′ boundary PCR product, indicated by an arrow in lane 4 of panel (A), was purified and cloned, and 16 clones were sequenced. The boxed nucleotides are not part of either the recombinant SART1 or the telomeric repeats.

FIG. 4 shows the necessity of ORFs and 3′UTR for SART1 retrotransposition. (A) is a schematic explanation of various mutant SART1-AcNPVs. The amino acid position of each missense mutation is shown. In the 1H626P mutant, for example, the histidine residue at the position 626 in ORF1 is substituted to proline. This position corresponds to the first histidine residue of the three continuous CCHC motifs in ORF1. The first methionine of the SART1 ORF1 is defined as the first, whereas in the case of ORF2, the amino acid residues preceding the first methionine in the overlapping ORF region are also counted. The mutant that lacks the entire 3′UTR and poly(A) sequence but still comprises the following polyhedrin 3′UTR is denoted as Δ3′. White arrows depict the pair of primers, +6096 and (CCTAA) 6, used for 3′ boundary amplification. (B) shows the 3′ boundary PCR assay in the Sf9 cells infected with only wild-type AcNPV or AcNPV comprising mutant SART1 elements (lane 1-7), or simultaneously coinfected with two kinds of mutant SART1-AcNPV (lane 8-14). The PCR products were subjected to 2% agarose electrophoresis and stained with ethidium bromide. The molecular size marker was run in the leftmost lane.

FIG. 5 shows that coinfected SART1 mutants, Δ3′ and 2C1007G, retrotranspose by trans-complementation. (A) shows the trans-complementation mechanism. ORF proteins derived from Δ3′act on 2C1007G SART1 RNA, and gives rise to the retrotransposed 2C1007G DNA. (B) shows an alternative possibility. DNA recombination near the ORF2 C-terminals between the two mutants generates wild-type SART1, which can subsequently retrotranspose. In (A) and (B), schematic structures of Δ3′ and 2C1007G are shown. Note that 2C1007G lacks the cysteine-histidine motif (indicated by a vertical line in the wild-type and Δ3′), but instead, comprises an additional ApaI site near the ORF2 C-terminal. White arrows depict the primers, +5616 and (CCTAA)₆, used for 3′ boundary PCR. The theoretical ApaI digestion fragments from the PCR products are shown as horizontal lines above the primers. (C) shows 3′ boundary PCR products undigested (lane 1 to 3) or digested with ApaI (lane 4 to 6). Molecular sizes are shown on the right.

FIG. 6 shows the target site alteration in a SART1/TRAS1 chimeric retrotransposon. (A) shows the schematic structures of SART1, TRAS1, and the SART1 with its APE replaced by TRAS1 APE. The portions derived from SART1 and TRAS1 are shown in gray and black, respectively. “RH” indicates the TRAS1 RNase H domain. White arrows represent the +6276 primer (for SART1 and the chimeric element), and TRAS1 +6022 primer, which are used in combination with (CCTAA)₆ or (TTAGG)₆. The deduced amino acid sequence for the N- and C-terminal boundaries of the APE domain is shown below. “AAAA” and “DLE” are derived from the linkers used for plasmid construction. The boundaries of the APE domain are based on a previous phylogenetic study (Malik, H. S. et al. (1999) Mol. Biol. Evol., 6, 793-805). (B) shows orientation-specific amplification of the 3′ boundaries of the three retrotransposons. The (CCTAA)₆ and (TTAGG)₆ primers used for PCR are denoted as “CCTAA” and “TTAGG”, respectively. (C) shows nucleotide sequences of the 3′ boundary PCR products in panel (B). Note that the 3′ boundary sequences of SART1 are described in FIG. 2B. The clone numbers are shown on the right.

FIG. 7 shows the production of a retroelement comprising a foreign gene. The foreign gene was inserted between ORF2 and the 3′UTR of SART1 (GST gene fused to ORF1) in an opposite orientation with respect to the retroelement. A plasmid encoding this retroelement was named T-sp.

FIG. 8 is a photograph showing the transposition of a retroelement comprising a foreign gene. A baculovirus was prepared from T-sp, and Bombyx BmN cells and Spodoptera Sf9 cells were infected with this baculovirus. Detection of the retroelement integrated into the chromosome by PCR confirmed that all cells incorporated the retroelement comprising the foreign gene. In BmN cells, low levels of transposition started to occur at around 24 hours, and maximum efficiency was reached at 96 hours. Meanwhile, in Sf9 cells, maximum introduction efficiency was observed at 72 hours.

FIG. 9 shows the construction of a vector (hsp pEGFP1-SART1 3′UTR) that incorporates the EGFP gene upstream of SART 3′UTR, which is expressed under the control of a Drosophila hsp promoter region (A), and the assay procedure for retrotransposition from this vector by trans-complementation. 24 hours after transfection of the hsp pEGFP1-SART1 3′UTR into Sf9 cells by lipofection, the cells were infected with AcNPV vector that had incorporated a 3′UTR-defective SART. DNAs were extracted 72 hours postinfection, and PCR was used to confirm whether transposition to the telomere had occurred.

FIG. 10 shows the result of investigating retrotransposition activity by a variety of SART1 3′UTR deletions. The numbers below 3′UTR indicate nucleotide positions. Note that since the recombinant SART1 used herein has a NotI site inserted adjacent to the 5′ end of 3′UTR, the nucleotide position is shifted by two nucleotides compared to the native SART1 3′UTR.

### BEST MODE FOR CARRYING OUT THE INVENTION

Herein below, the present invention will be specifically described using Examples, however, it is not to be construed as being limited thereto. All references cited herein are incorporated into this description.

### Example 1

### Plasmid Construction

The SART1 ORF1/ORF2/3′UTR portion was amplified by PCR from the genomic library clone, BS103 (Takahashi, H. et al. (1997) Nucl. Acids Res., 25, 1578-1584), using a pair of primers, SART1 S880 and SAX 3p Not1 (see Table 1). 30 cycles of PCR was conducted using Pfu Turbo™ DNA polymerase (Stratagene). The PCR product was subcloned between the NcoI and NotI sites of the pAcGHLTB plasmid (Pharmingen). The resulting plasmid, named SART1WT-pAcGHLTB, comprised the 64-bp polyhedrin 5′UTR and the GST-X₅-(His)₆-X₃₁-coding gene, SART1 ORF1 fused in-frame with MGSYKE--- of this gene (note that the underlined position is serine in the native SART1 ORF), followed by the SART1/ORF2/3′UTR, and the polyhedrin 3′UTR. Point mutations were introduced into SART1WT-pAcGHLTB with four pairs of primers listed in Table 1 using the QuickChange™ Mutagenesis Kit (Stratagene). The SART1 Δ3′-pAcGHLTB was constructed by digesting SART1WT-pAcGHLTB with AfIII and NotI, and ligating between these sites the 200-bp ORF2 3′ end sequence that had been amplified by PCR with the primers, SART1 S5995 and SART1 A6221. The mutation of each plasmid was confirmed by DNA sequencing. TRAS1WT-pAcGHLTB was constructed by cloning the TRAS1 ORF1/ORF2/3′UTR portion, which had been amplified from the genomic library clone, λB1 (Okazaki, S. et al. (1995) Mol. Cell. Biol., 15, 4545-4552) with a primer pair, TRAS1 S2395 and TRAS1 A7870, into the NcoI and NotI sites of pAcGHLTB plasmid. SART1-pAcGHLTB-comprising TRAS1 APE was constructed as follows: First, the NotI and BglII sites of SART1WT-pAcGHLTB were removed by NotI/BglII digestion, T4 DNA polymerase treatment, and self-ligation. Second, all but the APE domain of the SART1WT-pAcGHLTB was amplified by inverse PCR using the 5′-phosphorylated primers, SART1 A3029 and SART1 S3668. The amplified product was self-ligated and cloned. This construct, SART1 AAPE-pAcGHLTB, lacks the APE domain but instead comprises a NotI and a BglII site derived from the two primers. Third, the TRAS1 APE domain was amplified using TRAS1 S3848 and TRAS1 A4527, and cloned between the NotI and BglII sites of SART1 AAPE-pAcGHLTB.

TABLE 1

List of primers

| | | |
|---|---|---|
| Name | Sequence (5′ to 3′) | SEQ ID NO: |

| | | |
|---|---|---|
| +6276 | TGCCTACCTCACGAAGAAGTTGCGGTCA | 1 |
| +590 | ATTTTGGGAACGCATCCAGGCACATTGGGT | 2 |
| +6096 | AGAAAGAGAGTGCGACCCAAACTCAGTT | 3 |
| +5616 | AAGTGTGCCCCGTCTGTCTGTC | 4 |
| TRAS1 +6022 | GTAGTTAAGTATAGCGTAAGATATAGTCAGTAAG | 5 |
| SART1 S880 | AAAAAACCATGGGCAGTTATAAAGAAGAATTACCCCAG | 6 |
| SAX 3p Not1 | AAGGAAAAAAGCGGCCGCTTTTTTTTTTTTTTTTTTGG | 7 |
| SART1 S5995 | AGTCACTCGTCGCGGTG | 8 |
| SART1 A6221 | AAAAAAAAAAGCGGCCGCTACGGGAGCTGAGCG | 9 |
| SART1 1H626P | CACGCACTGGGGCC [symbol] CGTGAGTGCCCG | 10 |
| SART1 2H228V | GGAGACGCTCTCCGAC [symbol] CCGCTACATTGGTTTC | 11 |
| SART1 2D699V | GGTCATCTGCTACGCCG [symbol] CGACACGCTGGTGACG | 12 |
| SART1 2C1007G | GCCCTCGAAGCG [symbol] GCCCGAGGTGGG | 13 |
| TRAS1 S2395 | AAAAAACCATGGGACGCGTCCTCACTGCAA | 14 |
| TRAS1 A7870 | AATAATAATAGCGGCCGCTTTTTTTTTTTTTTTTTTTTAAGTCACTCTTTTCTCTGC | 15 |
| SART1 A3029 | TTTTTGCGGCCGCGCTGCTGGTCATTATTCGTCGTCCATTGGTGT | 16 |
| SART1 S3668 | AAAAAAAAGATCTGGAGTCTTCTTCGGTAACGACTTTGCCCTTTG | 17 |
| TRAS1 S3848 | AAAAAAAAAAGCGGCCGCCCCCTACAGAGTTTTGCAAG | 18 |
| TRAS1 A4527 | AAAAAAAGATCTTGGAGTCTAATATTGAATACCATACCG | 19 |

(The underlined letters indicate restriction enzyme recognition sites for subcloning. The boxed letters indicate mutated nucleotides.

### Example 2

### Recombinant AcNPV Generation

Sf9 cells were grown as monolayer cultures at 27° C. in TC-100 medium supplemented with 10% fetal bovine serum (Nihon-nosankougyou) in the presence of penicillin/streptomycin (Gibco). The recombinant baculovirus comprising the wild-type or mutant SART1 ORF1/ORF2/3′ UTR portion driven by the polyhedrin promoter was produced by co-transfection of the wild-type or mutant SART1-pAcGHLTB plasmid with the BaculoGold™ DNA (Pharmingen) into the Sf9 cells using the Tfx-20 lipofection reagent (Promega). Four days later, the medium was collected and used for plaque purification and subsequent virus propagation, according to the manufacturer's instructions (Pharmingen).

### Example 3

### Detection of In Vivo SART1 Retrotransposition by PCR Assay

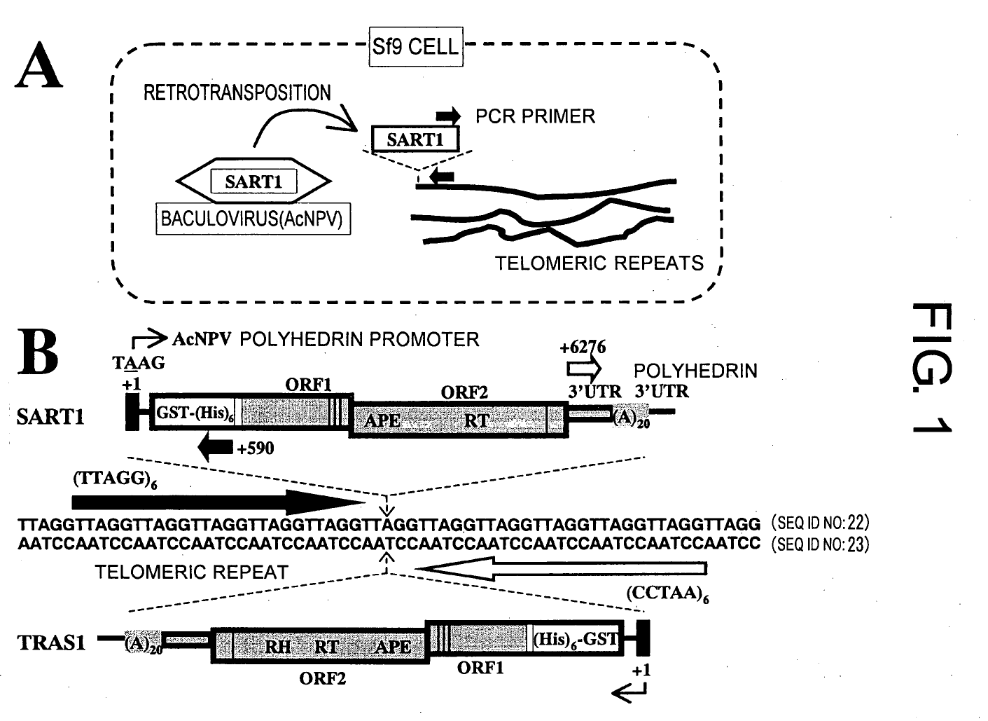

To detect in vivo SART1 retrotransposition, SART1 was expressed from AcNPV in Sf9 cells and this was monitored by PCR to see if the silkworm SART1 transposed into the Sf9 chromosomal telomeric repeats (FIG. 1A). In the recombinant AcNPV of Example 2, used in this heterologous expression system, the SART1 ORF1/ORF2/3′UTR portion is placed under the control of the AcNPV polyhedrin promoter (FIG. 1B, top). For future biochemical analysis, the SART1 ORF1 was fused to the C-terminal of GST-X₅-(His)₆-X₃₁ (X denotes the vector-derived amino acid) with the position of ORF2/3′UTR kept native relative to ORF1 (see Example 1). SDS-PAGE of the Sf9 total proteins confirmed that each virus expressed the putative GST-HiS₆-SART1 ORF1-fused protein, which is approximately 110 kDa in molecular weight (data not shown).

In vivo retrotransposition assays by PCR was performed as follows: Approximately 1×10⁶ Sf9 cells were infected in a 6-well plate with a SART1-comprising AcNPV at a multiplicity of infection (moi) of ten plaque forming units (pfu) per cell. As for the coinfection experiments described later, cells were infected with two AcNPVs at 5 pfu each per cell. At various hours post-infection (hpi), cells were scraped, pelleted by centrifugation at 1000 g for five minutes, washed twice with PBS at 4° C., and the total genomic DNAs were purified with a standard method using proteinase K and SDS (Ausubel, F. M. et al. (1994) Current Protocols in Molecular Biology, Greene Publishe Associates/John Wiley and Sons, New York. NY). The PCR assays were conducted with LA-Taq (Takara) in the presence of TaqStart Antibody (Clontech) using approximately 10 ng of Sf9 DNA. The reaction solution was denatured at 94° C. for three minutes, followed by 35 cycles (for the SART1 3′boundary) or 40 cycles (for the SART1 5′ boundary, TRAS1 3′ boundary, and SART1/TRAS1 APE 3′ boundary) of 98° C. for 20 seconds, 62° C. for 30 seconds, and 72° C. for one minute. Ten microliters from each mixture was subjected to 2 or 3% agarose-gel electrophoresis in TBE buffer and visualized by ethidium-bromide staining. PCR products were cloned into the pGem-T-easy vector (Promega), after being excised directly or using RECOCHIP (Takara) from the agarose gel. The cloned products were sequenced using Big Dye Terminator Cycle Sequencing Kit (Applied Biosystems) on an automatic DNA sequencer, ABI310 Genetic Analyzer. Sequence analysis was carried out using DNASIS-Mac version 3.7 (Hitachi).

### Example 4

### The 3′ Boundary Between the Retrotransposed SART1 Elements and the Telomeric Repeats is Identical to that Found in the Bombyx Genome

First, the Sf9 cells were infected with the recombinant SART1-AcNPV. 7, 24, 48, and 72 hours postinfection (hpi), the cells were pelleted by centrifugation, washed, and the Sf9 total genomic DNAs were extracted. The purified DNA was subjected to PCR to amplify the boundaries between transposed SART1 elements and the Sf9 telomeric repeats. To amplify the 3′ boundary, the +6276 primer complementary to SART1 3′UTR (Table 1), and the (CCTAA) 6 primer (SEQ ID NO: 20) were used (FIG. 1B, top and middle). Likewise, for the 5′ boundary the +590 primer complementary to the GST gene coding strand, and the (TTAGG)₆ primer (SEQ ID NO: 21) were used.

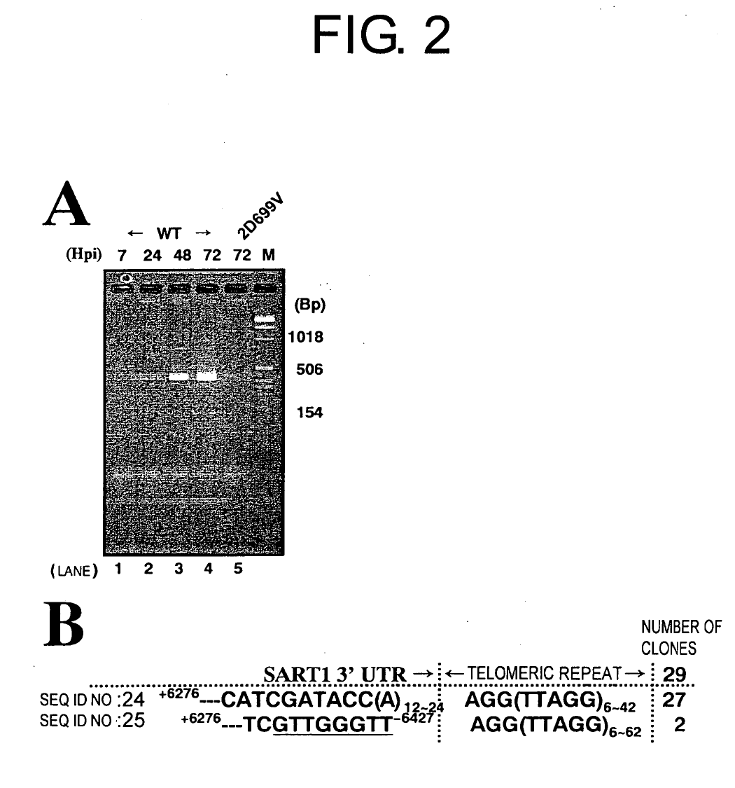

Surprisingly, with only 35 cycles of the 3′ boundary PCR, an intense band was observed 24 to 72 hours post-infection (hpi), suggesting highly efficient transposition in this system (FIG. 2A). The observed time course accurately reflects the polyhedrin promoter expression because the polyhedron promoter is activated 20 to 24 hpi (O'Reilly, D. R. et al. (1992) Baculoviral Expression Vectors: A Laboratory Manual. W.H. Freeman and Company, NY). The size, approximately 400 bp, is in good accordance with that of the putative retrotransposed 3′ boundary, 392 bp plus telomeric repeat length. Total PCR products in lane 4 were cloned into a plasmid vector, and 29 clones were sequenced (FIG. 2B). All 29 clones were amplified correctly by the +6276 and (CCTAA) 6 primers. Among them, 27 comprised full-length 3′UTRs with poly(A) tails connected with the telomeric repeats. Importantly, the poly(A) tails of all 27 clones were directly linked to the AGG of the telomeric repeats, similarly to the boundary sequences found in the Bombyx genome (Takahashi, H. et al. (1997) Nucl. Acids Res., 25, 1578-1584). These results suggest that these 27 SART1 clones arose from retrotransposition.

The other two clones, however, comprised only the 5′-half 152 bp of the SART1 3′UTR. They were linked to the telomeric repeats at an octanucleotide, GTTGGGTT (underlined nucleotides in FIG. 2B). Since this octamer sequence is only one-base different from the telomeric repeat, GTTAGGTT, these two SART1 clones may have arisen by recombinational events with endogenous Sf9 telomeric repeats. Transduction of 3′ flanking sequences, often found in human L1, was not observed, (Moran, J. V. et al. (1999) Science, 283, 1530-1534).

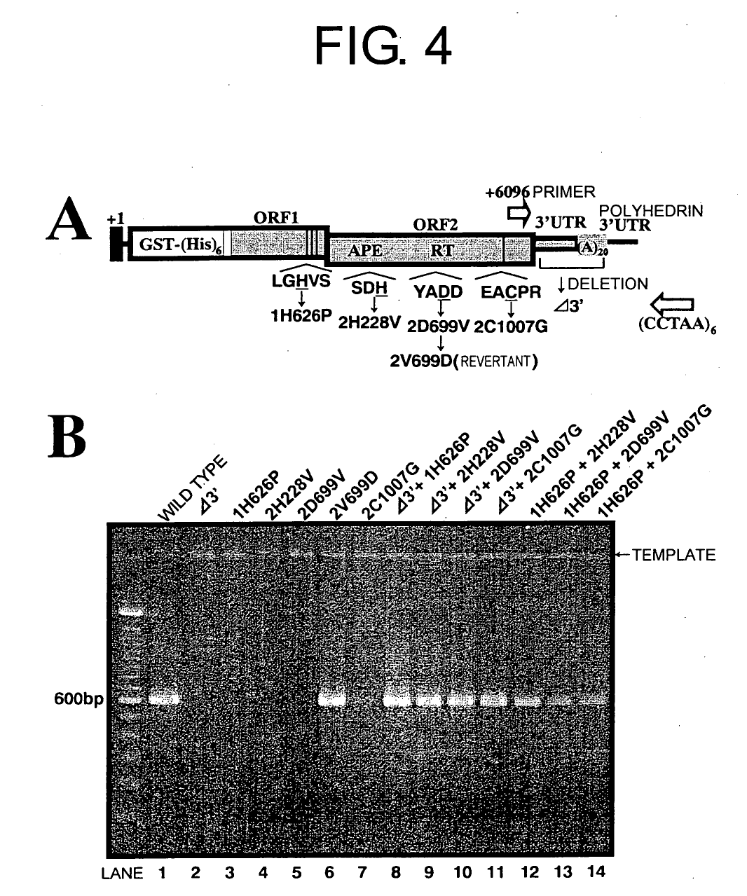

As a negative control, Sf9 cells were infected with SART1 2D699V-AcNPV, the mutant of the putative SART1 reverse transcriptase C motif active site, YADD. In this mutant, the aspartic acid residue at the ORF2 amino acid position 699 was substituted to a valine residue (FIG. 4A). A PCR assay for this mutant did not detect any retrotransposition (FIG. 2A, lane 5). This result indicates that the detected transposition was not mediated by endogenous Sf9 SART-like elements, but by authentic retrotransposition of the B. mori SART1 by its own RT activity.

### Example 5

### Retrotransposition of SART1 is Mediated by RNA

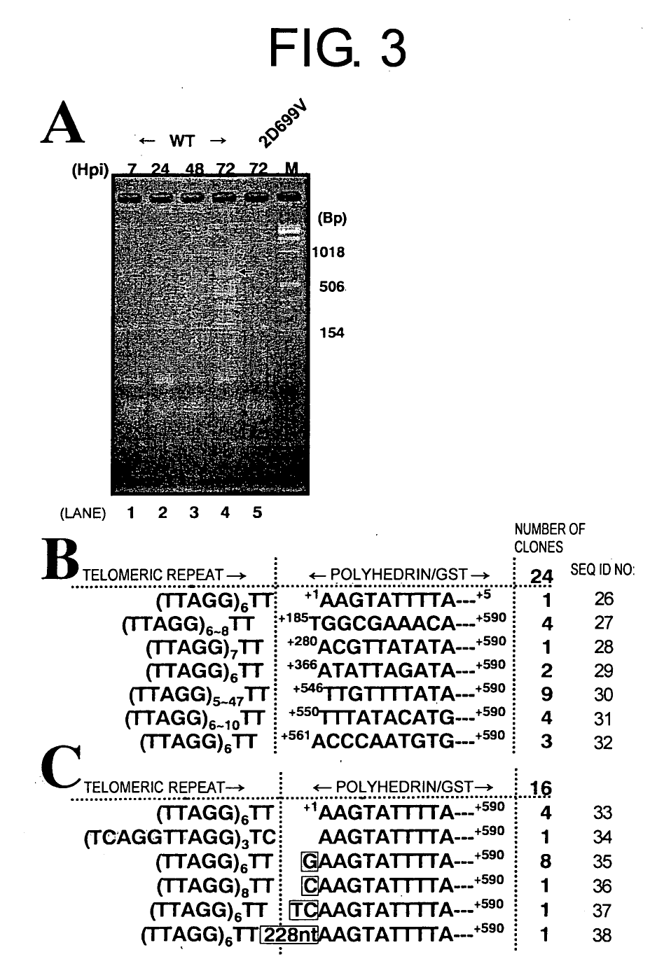

The amplification of the 5′ boundary through 40 cycles of PCR gave rise to visible bands at 72 hpi (FIG. 3A). In contrast to the 3′ boundary, several bands appeared. The size of the largest band (arrow in lane 4), approximately 600 bp, was in good accordance with the putative full-length 5′ transposed product length, 590 bp plus the telomeric repeat (FIG. 1B). The present inventors therefore predicted that this band represented full-length retrotransposition and the smaller bands are 5′ deletions arising from abortive reverse transcription. Cloning and subsequent sequencing of the whole PCR products in lane 4 confirmed that the present inventors' prediction was correct (FIG. 3B). All 24 sequenced clones were amplified by the (TTAGG)₆ and +590 primers. In all of the clones, the transposed SART1 5′ ends were connected 3′ to the TT of (TTAGG)ₙ, the same insertion position as in the Bombyx genome. In the largest clone, the telomeric repeat was precisely linked with the polyhedrin RNA 5′ end sequence, AAG (FIG. 1B; Possee, R. D. and Howard, S. C. (1987) Nucl. Acids Res., 15, 10233-10248). This result strongly implies that the recombinant SART1 was transposed through RNA. In all of the other 23 clones, it turned out that SART1 elements with diversely deleted 5′ ends were connected with the telomeric repeats. None of the 5′ bands were detected from the cells infected with SART1 2D699V-AcNPV (FIG. 3A, lane 5). Successful detection by 5′ boundary PCR also suggests that first strand synthesis was followed by an integration step between the 5′ sequence of the reverse transcribed first strand DNA and the (CCTAA)ₙ, and/or second strand DNA synthesis primed by (TTAGG).

In the Bombyx genome, the present inventors have previously found examples of duplication and aberration at the SART1 DNA 5′ ends (see FIG. 4 in Takahashi, H. et al. (1997) Nucl. Acids Res., 25, 1578-1584). To examine whether similar aberrant 5′ sequences would be observed, the present inventors analyzed the full-length retrotransposition products extracted from the largest band in lane 4 of FIG. 3A (indicated with an arrow). Subcloning and sequencing of the 16 clones showed that the polyhedrin RNA 5′ end sequence, AGG, was directly linked to TT of the telomeric repeats in four clones (FIG. 3C). This represents normal full-length retrotransposition. In another clone, SART1 retrotransposed into 10-mer repeats, (TCAGGTTAGG)ₙ, which is only one nucleotide different from the telomeric repeat unit. Eight of the other ten clones had an extra guanidine (G) between the recombinant SART1 elements and the telomeric repeats. There was one case each of an extra C or TC. The G may arise commonly as a result of reverse transcription of the 5′ G cap (Hirzmann, J. et al. (1993) Nucl. Acids Res., 21, 3597-3598; Volloch, V. Z. et al. (1995) DNA Cell Biol., 14, 991-996). Alternatively, these added nucleotides may represent terminal deoxynucleotidyl transferase activity of the SART1 RT. In the other clone, a 228-bp unknown sequence was added, which is difficult to explain. Although these variations were somewhat different from those found in the Bombyx genome, the existence of the 5′ deletion and aberration also supports the normal retrotransposition of SART1 in this system.

### Example 6

### SART1 Retrotransposition Requires the 3′UTR and Conserved Motifs in Both ORFs

SART1 is a typical LINE with two ORFs (ORF1 and ORF2). ORF1 comprises three C-terminal cysteine-histidine motifs, and ORF2 comprises an APE, an RT domain, and a C-terminal cysteine-histidine motif (FIG. 1B). To examine whether these conserved motifs are essential for SART1 to retrotranspose in vivo, the present inventors generated a series of SART1-AcNPV constructs comprising missense mutations in these conserved motifs, and assayed to determine whether these elements could transpose into the telomeric repeats (FIG. 4A). The present inventors also made a SART1 Δ3′-AcNPV construct, which lacks the entire SART13′UTR but retains a downstream polyhedrin 3′UTR. For these elements, a 3′ boundary PCR assay was conducted using the (CCTAA) 6 primer and +6096 primer complementary to the SART1 ORF2 (Table 1).

As shown in FIG. 4B, none of these mutants could transpose in vivo (lanes 2 to 5, and 7). This result indicates that the APE and RT domains, and the cysteine-histidine motif in ORF2, are indispensable for in vivo SART1 retrotransposition. Disruption of the ORF1 cysteine-histidine motifs also blocked retrotransposition. This result shows that the ORF1 cysteine-histidine motifs, which are widely conserved from many LINEs to retroviruses, are essential for retrotransposition. The SART1 retrotransposition also required 3′UTR, suggesting that the sequence-specific recognition of the RNA 3′ end by the ORF proteins is essential for SART1 to retrotranspose. Since SART1 Δ3′ construct with a remaining polyhedron 3′UTR was not retrotransposed, SART1 is unlikely to recognize only the poly(A) tail. Because SART1 5′UTR was replaced by the polyhedrin 5′UTR in the construct, it is shown to be unnecessary for retrotransposition.

These mutant SART1-AcNPVs were constructed by a two-step procedure: plasmid mutagenesis and virus generation. The present inventors confirmed that each mutant expressed a comparable amount of the putative SART1 ORF1 protein (data not shown). However, these mutant SART1 elements may have failed to retrotranspose because undesired deleterious mutations were introduced into other amino acid positions during the two steps. To exclude this possibility, the present inventors conducted two control experiments. First, as a control for plasmid mutagenesis, the valine residue in the 2D699V-AcGHLTB was re-mutated to an aspartic acid (FIG. 4A, 2V699D). The resulting plasmid should have a nucleotide sequence identical to the wild-type SART1. The ACNPV made from this plasmid restored wild-type level of retrotransposition (FIG. 4B lane 6), indicating that the retrotransposition deficiency in the 2D699V mutant was not due to any possible undesired mutations during plasmid mutagenesis.

As another control, the present inventors performed coinfection with two of these mutant viruses, and assayed to determine whether retrotransposition occurred. If these mutants did not have unintended mutations, other than those introduced by the present inventors, the two infected mutants might supply the ORF proteins and the RNAs to each other, resulting in retrotransposition by trans-complementation. As anticipated, coinfection enabled SART1 retrotransposition (FIG. 4B lanes 8 to 14). Approximately wild-type-level signals were detected from the Δ3′ mutant coinfected with each of the ORF mutants (lanes 8 to 11). This result suggests that the Δ3′ mutant still expresses functional ORF proteins that can act efficiently on the RNA 3′ end derived from each ORF mutant. Similarly, since retrotransposition of a somewhat reduced level was observed in the ORF1 mutant, 1H626P, which was coinfected with each of the ORF2 mutants (lanes 12 to 14), it is suggested that 1H626P correctly produced the functional ORF2 protein and retrotransposition was accomplished by trans-complementation with the ORF1 protein supplied from each ORF2 mutant. These analyses suggest that retrotransposition deficiency in each mutant was not caused by experimental errors during the mutant AcNPV construction, but by the effect of the mutations introduced by the present inventors.

Furthermore, in the experiment in which the SART1 Δ3′mutant was coinfected with the ORF mutants, if SART1 only recognizes the poly(A) tail, the SART1 ORF protein would bind in trans to more poly(A) of cytoplasmic mRNA than SART1 RNA, and efficient retrotransposition of SART1 Δ3′ mutants would seem unlikely. However, efficient retrotransposition of the SART1 Δ3′ mutant was actually observed, indicating that an SART1 3′UTR portion other than the poly(A) tail is important for SART1 retrotransposition.

### Example 7

### Retrotransposition by Trans-Complementation

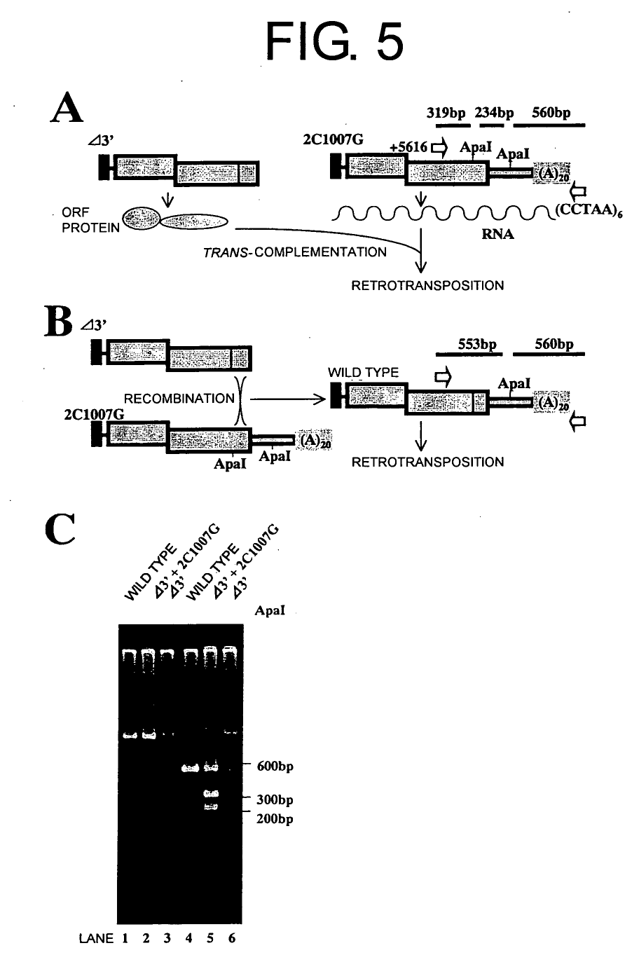

The results presented above suggest that SART1 can retrotranspose by delivering its encoding proteins in trans to other SART1 RNAs or protein molecules. There remains a less likely possibility, however, that the retrotransposition was subsequently caused by the wild-type SART1 element generated through recombination between two mutant DNAs. To rule out this possibility, a 3′ PCR product derived from the coinfection of the Δ3′ mutant and 2C1007G mutant was analyzed (see FIG. 4B, lane 11). The size of the product suggests that only the products having the same length as wild-type were transposed, and not the Δ3′ elements. In the SART1 2C1007G-pAcGHLTB construction by plasmid mutagenesis, the present inventors introduced an ApaI restriction enzyme recognition site in the 2C1007G mutant. If retrotransposition occurred through reverse transcription of the 2C1007G RNA by trans-complementation, the transposed DNA 3′ end should have an additional ApaI site at the mutagenized position in addition to the ApaI site in the 3′UTR (FIG. 5A). On the other hand, if the retrotransposition was subsequently caused by the wild-type SART1 generated by homologous recombination, the retrotransposed DNA product would have only one ApaI site in the 3′UTR (FIG. 5B). Thus, a 3′ boundary PCR was performed using the (CCTAA)₆ primer and +5616 primer complementary to ORF2 (FIG. 5C). A 1.1-kb band was detected from the Sf9 cells infected with the wild-type SART1 (lane 1) or with both of the two mutants simultaneously (lane 2). The ApaI digestion of the wild-type PCR product gave rise to two bands of approximately 550 bp (lane 4), whereas digestion of the PCR product from a double infection mutant gave three bands, as expected from trans-complementation (lane 5). The amplification from cells infected solely with Δ3′ did not produce the band that could be digested with ApaI (lanes 3 and 6). These experiments suggest that the SART1 retrotransposition observed with coinfection of two mutants is not due to re-generation of a wild-type SART1 by DNA recombination, but results from trans-complementation between the two mutant SART1 elements. The absence of a 3′ deleted product suggests that the Δ3′ mutants lack an essential cis element required for transposition.

### Example 8

### Exchanging the APE Domains Between LINEs Alters the Insertion Site Specificity

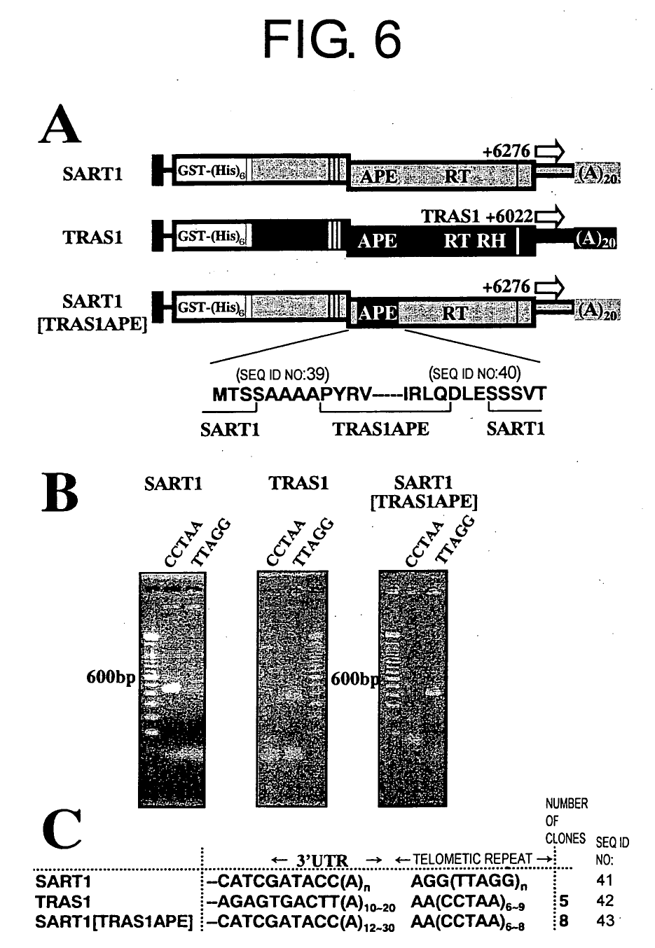

An indispensable step in LINE retrotransposition is the nicking of target site DNAs, and these DNAs are thought to serve as primers for reverse transcription. Because the APE domain protein expressed in bacteria cleaves oligonucleotides comprising the target site sequences in vitro (Feng, Q. et al. (1996) Cell, 87, 905-916), this domain may be responsible for target cleavage. Although an APE domain was important for in vitro target DNA cleavage (Feng, Q. et al. (1996) Cell, 87, 905-916), this proposed function of an APE domain has not been proved in the context of in vivo retrotransposition. Thus, the present inventors developed a novel approach using the system of the present invention. TRAS1 is another retrotransposon, which is inserted at a specific nucleotide position with the opposite orientation to SART1 relative to the telomeric repeats (FIG. 1B; Okazaki, S. et al. (1995) Mol. Cell. Biol., 15, 4545-4552). Utilizing the insertion sequence differences of these two elements, a chimeric SART1-TRAS1 APE element was constructed, in which the SART1 APE domain was replaced by the TRAS1 APE domain and the other SART1 portions was kept native (FIG. 6A). If the TRAS1 APE domain determines the target site of this chimeric retrotransposon, this element would be inserted at the same nucleotide position as TRAS1, but not as SART1 within the telomeric repeats.

First, whether SART1 was inserted into the telomeric repeats in a specific orientation relative to the telomeric repeats was examined. A 3′ PCR assay was conducted using the +6276 primer, in combination with either the (TTAGG)₆ or (CCTAA)₆ primer (FIG. 6B). As expected from the insertion orientation of SART1, a band was detected when using the (CCTAA) 6 primer but not when using the (TTAGG) 6 primer.

Next, whether TRAS1 could retrotranspose in vivo and whether the TRAS1 insertion exhibits the opposite orientation specificity to SART1 were investigated. The TRAS1 ORF1/ORF2/3′UTR portion was cloned downstream of the polyhedrin promoter in the pAcGHLTB plasmid and an AcNPV expressing TRAS1 was generated (FIG. 1B, bottom). A 3′PCR assay was carried out using the TRAS1 +6022 primer complementary to TRAS1 3′UTR, in combination with either one of the (TTAGG) 6 and (CCTAA) 6 primers (FIG. 6B). In contrast to SART1, the band was detected when using the (TTAGG) 6 primer but not when using the (CCTAA) 6 primer. This band was cloned and sequenced (FIG. 6C). In all five clones, the 3′ end of TRAS1 was adjacent to the telomeric repeats with the poly(A) tails bound 5′ to the AA of (CCTAA)ₙ. This insertion position is exactly identical to that observed in the Bombyx genome. The retrotransposition was blocked when conserved amino acid residues were mutated (data not shown). Therefore, TRAS1 is also retrotransposition-competent and has the opposite insertion orientation specificity to SART1.

The present inventors then constructed the chimeric SART1-TRAS1 APE element, which was assayed with 3′ PCR using the +6276 primer complementary to the SART1 3′UTR, in combination with either one of the telomeric repeat primers. As shown in FIG. 6B, this element showed the same insertion orientation as TRAS1 but opposite to SART1. Cloning and subsequent sequencing of the PCR products demonstrated that, in all eight sequenced clones, this element inserted at the exact same nucleotide position as TRAS1 (FIG. 6C). This result provided in vivo evidence that the APE domain is the primary determinant for target site selection in LINE retrotransposition.

### Example 9

### Production and Retrotransposition of a Retroelement Comprising a Foreign Gene

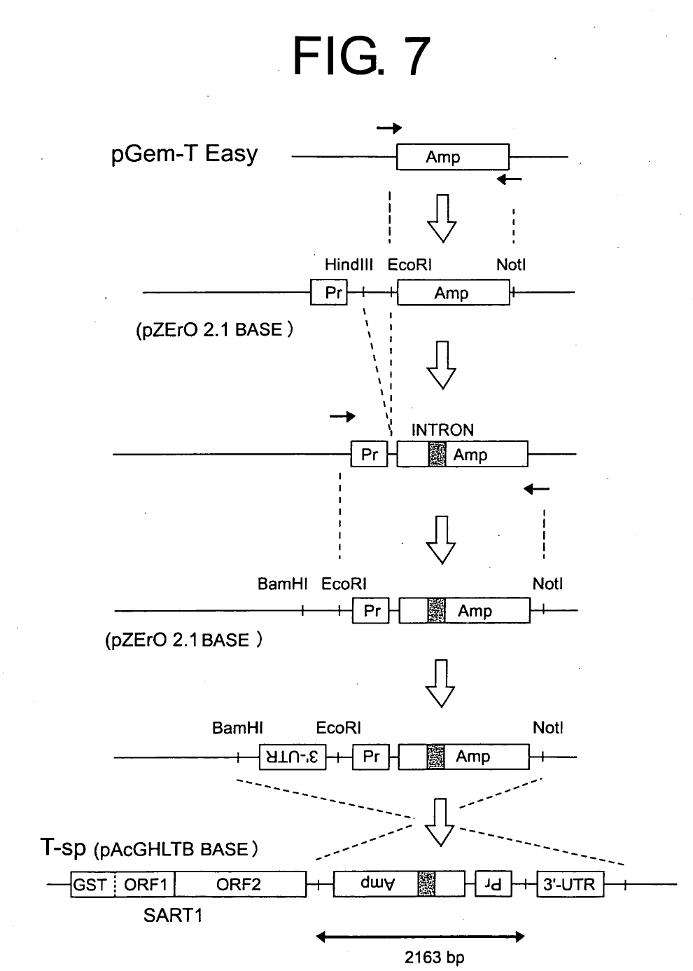

As a foreign gene, the Amp region of a plasmid, pGEM-T EASY (Promega), was amplified by PCR, and the amplified product was integrated into the EcoRI/NotI site of pZEr0-2.1 (Invitrogen). Digestion of the obtained plasmid with HindIII/EcoRI and self ligation removed the HindIII and EcoRI sites. An intron derived from silkworm actin gene was also inserted into the Amp-encoding region. A fragment comprising the region from Ori to the Amp gene was PCR amplified using the obtained plasmid as a template, and the amplified fragment was integrated into the EcoRI/NotI site of pZEr0-2.1. BamHI/EcoRI fragment comprising the SART1 full-length 3′UTR (SEQ ID NO: 52) was inserted into this plasmid. The fragment comprising SART1 full-length 3′UTR was PCR amplified using a primer pair, SART1S6221EcoRI (5′-ttttttgaat tcggaccgtc gggcgtc-3′/SEQ ID NO: 53) and SART1A6704BglIIBamHI (5′-ttttttggat ccagatcttt tttttttttt tttttttggt atcga-3′/SEQ ID NO: 54), comprising EcoRI and BamHI restriction enzyme sites, respectively. The NotI/BamHI fragment comprising the region from the Amp gene to 3′UTR was excised and this was introduced immediately after the ORF2 stop codon of a baculovirus transfer vector, pAcGHLT B (PharMingen), which encodes the ORF1 (GFP-fused) and ORF2 of SART1, and the obtained plasmid was then named T-sp (FIG. 7).

Ultimately, a 2163-bp gene fragment was incorporated into this plasmid as a foreign gene not derived from SART. Sf9 cells and BmN4 cells were infected with a baculovirus produced from this plasmid, and using the combination of the SART internal primer (Tsp-S10377, S8499, 10098) with the primer, (CCTAA)₅T₅, in the telomeric repeat sequence, PCR analysis determined whether the sequences introduced to the cells were inserted into the telomeric repeat sequences in the chromosomes.

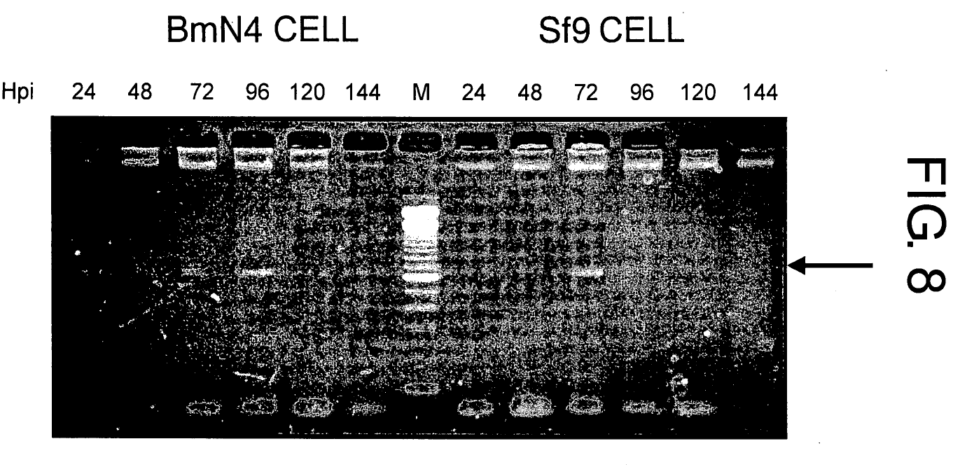

PCR analyses confirmed that both Bombyx BmN cells and Spodoptera Sf9 cells incorporated SART comprising a foreign gene, as shown in FIG. 8. This result verifies the broad range of hosts of AcNPV, and suggests introduction into broader range of animal cells. In BmN cells, low level of transposition started to occur at approximately 24 hours, and maximum efficiency was reached at 96 hours. On the other hand, in Sf9 cells maximum introduction efficiency was observed at 72 hours. This result shows that a foreign gene of at least 2.5 kb or so can be introduced into the genome by this method. As indicated in Example 11, approximately 70 bases at the 5′-side region of SART1 3′UTR, and approximately 80 bases at the 3′ region are not essential for the transposition. Therefore, the 3′ region also becomes a candidate for a site to introduce a foreign gene. Since reverse transcription occurs from the 3′ side to the 5′ side in non-LTR retrotransposons, truncation often occurs on the 5′ side. Because insertion of a foreign gene farthest downstream of the retrotransposon as in FIG. 7 reduces the risk of truncation at the 5′ side, this is advantageous in that even if a huge foreign gene is introduced, truncation is barely possible in at least that portion during transposition.

### Example 10

### Retrotransposition of a Foreign Gene with a 3′UTR Region, by Utilizing Trans-Complementation

(Method)

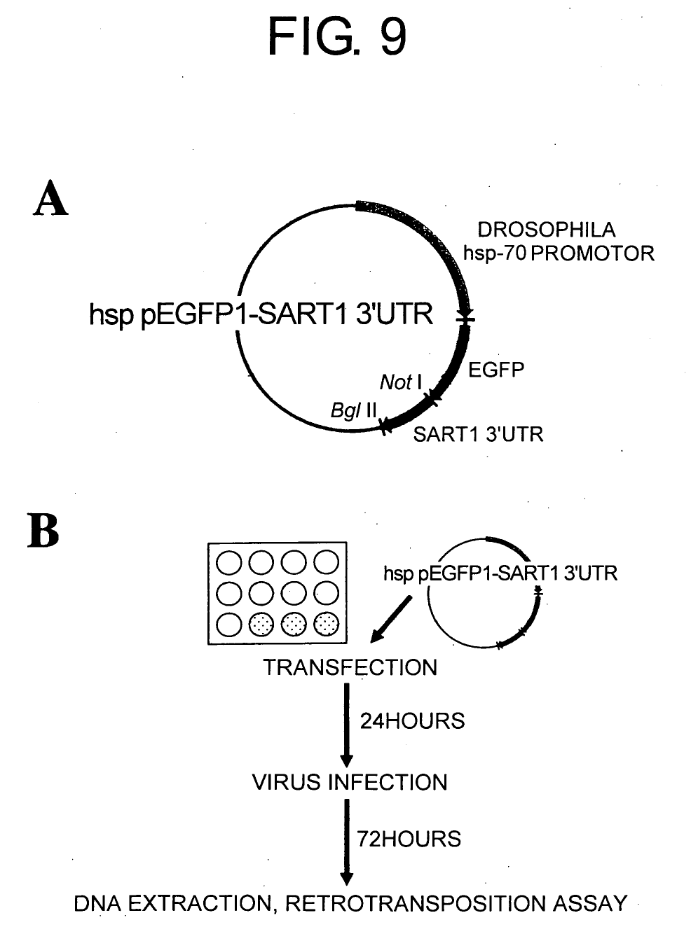

A vector where an EGFP region expressed under the control of a Drosophila hsp promoter region has been integrated upstream of the full-length 3′UTR of SART (hsp pEGFP1-SART1 3′UTR) (FIG. 9) was produced. 24 hours after transfecting this plasmid to Sf9 cells by lipofection, the cells were infected with AcNPV vector into which 3′UTR-deficient SART (SART1Δ3′-AcGHLTB) has been integrated. DNAs were extracted 72 hours postinfection, and PCR confirmed whether transposition to the telomere occurred. Two types of infection, infection with the plasmid alone or with a reverse transcriptase-deficient strain (SART1 2D699V-AcGHLTB), were used as the control.

(Results)

The DNAs from the cells coinfected with hsp pEGFP1-SART1 3′UTR and SART1 Δ3′-AcGHLTB were used as templates for PCR, detected bands were excised, and sequence determination was performed. The result showed that all 22 clones were inserted into the telomeric repeat sequence (Table 1). Therefore, the 3′UTR in the plasmid was recognized by SART protein expressed by the baculovirus, and the upstream portion thereof may have transposed due to reverse transcription. This result demonstrated that just by transfecting a plasmid, a foreign gene can be easily transposed to a genomic target site by utilizing trans-complementation. Reverse transcription did not take place from the 3′-end of 3′UTR, but from the site spanning from the middle to the latter half of the 3′UTR region. Specifically, most of the reverse transcription occurred from position 6462 in the latter half of 3′UTR, and this site is predicted to be involved in recognition for reverse transcription initiation. This result is very similar to that of LINE transposition when polyA is deficient. Thus, when proteins of the retroelement are acted in trans to 3′UTR-carrying plasmids, the mechanism may be different from that of complete LINEs comprising polyA, for example, the reverse transcriptase initiation complex may recognize a different region. Transposition to a telomeric repeat sequence was not observed in the controls, which were transfection experiments using only the plasmid, or coinfection experiments with a reverse transcriptase-deficient SART.

TABLE 2

The 3′-boundary of EGFP-SART1 3′ UTR

| | | | |
|---|---|---|---|
| | | | SEQ |
| | | Number of | ID |
| EGFP1-SART1 3′UTR | Telomeric repeat seqeuce | clones: 22 | NO: |

| | | | |
|---|---|---|---|
| ⁺⁶⁹⁴----TGGTGGTGAG⁺⁶²⁸⁷ | T (TTAGG)₆ | 1 | 44 |
| ⁺⁶⁹⁴----GGTGGTGAGG⁺⁶²⁸⁸ | AGG (TTAGG)ₙ | 4 | 45 |
| ⁺⁶⁹⁴----AGGGTATAGG⁺⁶²⁹⁵ | AGG (TTAGG)₅ | 1 | 46 |
| ⁺⁶⁹⁴----GTATAGGGCG⁺⁶²⁹⁸ | AGG (TTAGG)ₙ | 2 | 47 |
| ⁺⁶⁹⁴----GGAGCTCGTT⁺⁶⁴⁵⁷ | AGG (TTAGG)₅ | 1 | 48 |
| ⁺⁶⁹⁴----TGGGCGGGTT⁺⁶⁴⁹² | AGG (TTAGG)₆ | 1 | 49 |
| ⁺⁶⁹⁴----TCGTTGGGTT⁺⁶⁴⁶² | AGG (TTACG)ₙ | 12 | 50 |

### Example 11

### Retrotransposition Activity of a 3′UTR-Deficient Mutant of SART1

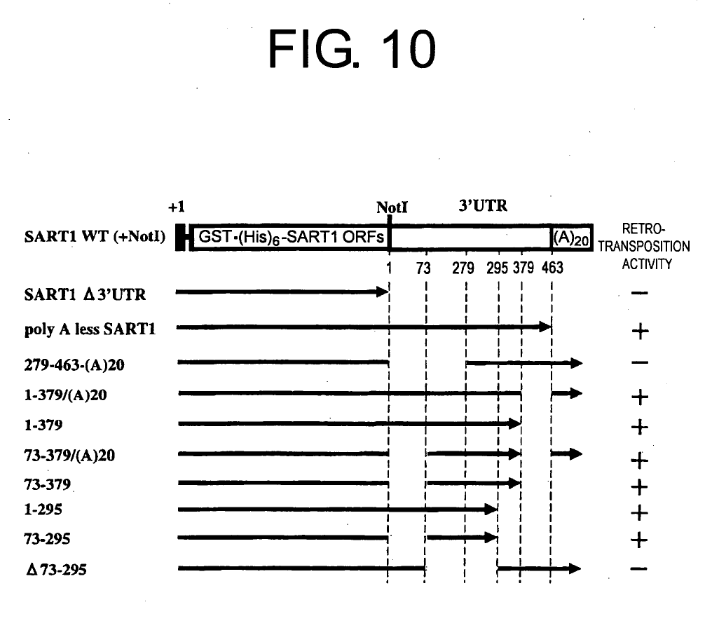

In order to search for the 3′UTR region necessary for retrotransposition, plasmids were constructed with a variety of deletions in the GST-(His)₆-fused SART1 3′UTR of Example 1, as shown in FIG. 10. Recombinant AcNPV was generated from these plasmids, as in Example 2, and retrotransposition assays were performed as in Example 3. Although retrotransposition activity was maintained even when the polyA (A₂₀) downstream of 3′UTR was deleted, transposition efficiency was significantly decreased. As indicated in FIG. 10, since retrotransposition occurred even after deleting 84 or 168 nucleotides from the 3′ end of 3′ UTR, and retrotransposition also occurred even after deleting 71 nucleotides from the 5′ end of 3′UTR, these sequences were shown to be unessential for transposition. However, retrotransposition activity disappeared when nucleotides 71 to 293 were deleted from the 5′ end of SART1 3′UTR. Furthermore, since RNA comprising the nucleotide sequence from the 71st to the 293rd nucleotide at the 5′ end of 3′UTR was able to retrotranspose, the sequence essential for retrotransposition activity was suggested to be comprised in this region.

### INDUSTRIAL APPLICABILITY

The present invention utilizes LINE retrotransposition by trans-complementation to enable efficient introduction of nucleic acids to chromosomes in cells. Replacement of LINE endonuclease domains with the endonuclease domains of target-specific LINEs allowed target specificity to be imparted to LINEs. Gene transfer vectors of target-specific LINEs, constructed based on viruses, retrotranspose very efficiently to host chromosomes. The retrotransposition systems of the present invention enable gene delivery with little harm to the host.

1. A method for retrotransposing an RNA, wherein the method comprises the steps of
(i) transcribing an RNA in a cell, wherein the RNA comprises a 3′UTR fragment of a LINE, and
(ii) expressing an ORF protein of the LINE, from somewhere other than the RNA.

2. The method of claim 1, wherein the LINE is an APE domain-comprising LINE.

3. The method of claim 1, wherein the LINE is a site-specific LINE.

4. A method for retrotransposing an RNA, wherein the method comprises the steps of
(i) transcribing an RNA in a cell, wherein the RNA comprises a 3′UTR fragment of an APE domain-comprising site-specific LINE, and
(ii) expressing an ORF protein of the LINE in the cell.

5. A method for retrotransposing an RNA, wherein the method comprises the steps of
(i) transcribing an RNA in a cell, wherein the RNA comprises a 3′UTR fragment of a LINE, and
(ii) expressing an ORF protein of the LINE in the cell, wherein the endonuclease domain of the ORF protein has been replaced with an endonuclease domain of another LINE.

6. The method of claim 5, wherein the other LINE is an APE domain-comprising LINE.

7. The method of claim 5, wherein the other LINE is a site-specific LINE.

8. The method of any one of claims 3, 4, and 7, wherein the site-specific LINE is a telomeric repeat-specific LINE.

9. The method of claim 8, wherein the telomeric repeat-specific LINE is a member of TRAS family or SART family.

10. The method of any one of claims 1 to 9, wherein the ORF protein and/or the RNA is expressed from a viral vector.

11. A retrotransposition vector encoding an RNA comprising a 3′UTR fragment of a LINE, wherein the vector does not express an ORF protein encoded by the LINE.

12. A vector encoding an ORF protein encoded by a LINE, wherein the endonuclease domain of the protein has been replaced with an endonuclease domain of an ORF protein encoded by a site-specific LINE.

13. The vector of claim 11 or 12, wherein the vector is a viral vector.

14. The viral vector of claim 13, wherein the virus does not integrate into chromosomes.

15. The viral vector of claim 14, wherein the virus that does not integrate into chromosomes is a baculovirus.

16. A kit for gene delivery mediated by retrotransposition of an RNA, wherein the kit comprises
(i) a vector expressing an ORF protein encoded by a LINE, and
(ii) a vector that encodes an RNA comprising a 3′UTR fragment of the LINE, and which does not express the ORF protein.

17. The kit of claim 16, wherein the ORF protein comprises an endonuclease domain of an ORF protein encoded by a site-specific LINE.

18. The kit of claim 17, wherein the vector is a viral vector.


SEQUENCE LISTING (54 sequences)

SEQ ID NO: 1 - DNA, 28 bp, Artificial Sequence
  artificially synthesized primer sequence
tgcctacctcacgaagaagttgcggtca

SEQ ID NO: 2 - DNA, 30 bp, Artificial Sequence
  artificially synthesized primer sequence
attttgggaacgcatccaggcacattgggt

SEQ ID NO: 3 - DNA, 28 bp, Artificial Sequence
  artificially synthesized primer sequence
agaaagagagtgcgacccaaactcagtt

SEQ ID NO: 4 - DNA, 22 bp, Artificial Sequence
  artificially synthesized primer sequence
aagtgtgccccgtctgtctgtc

SEQ ID NO: 5 - DNA, 34 bp, Artificial Sequence
  artificially synthesized primer sequence
gtagttaagtatagcgtaagatatagtcagtaag

SEQ ID NO: 6 - DNA, 38 bp, Artificial Sequence
  artificially synthesized primer sequence
aaaaaaccatgggcagttataaagaagaattaccccag

SEQ ID NO: 7 - DNA, 38 bp, Artificial Sequence
  artificially synthesized primer sequence
aaggaaaaaagcggccgcttttttttttttttttttgg

SEQ ID NO: 8 - DNA, 17 bp, Artificial Sequence
  artificially synthesized primer sequence
agtcactcgtcgcggtg

SEQ ID NO: 9 - DNA, 33 bp, Artificial Sequence
  artificially synthesized primer sequence
aaaaaaaaaagcggccgctacgggagctgagcg

SEQ ID NO: 10 - DNA, 26 bp, Artificial Sequence
  artificially synthesized primer sequence
cacgcactgggccccgtgagtgcccg

SEQ ID NO: 11 - DNA, 34 bp, Artificial Sequence
  artificially synthesized primer sequence
ggagacgctctccgacgtccgctacattggtttc

SEQ ID NO: 12 - DNA, 34 bp, Artificial Sequence
  artificially synthesized primer sequence
ggtcatctgctacgccgtcgacacgctggtgacg

SEQ ID NO: 13 - DNA, 25 bp, Artificial Sequence
  artificially synthesized primer sequence
gccctcgaagcgggcccgaggtggg

SEQ ID NO: 14 - DNA, 30 bp, Artificial Sequence
  artificially synthesized primer sequence
aaaaaaccatgggacgcgtcctcactgcaa

SEQ ID NO: 15 - DNA, 57 bp, Artificial Sequence
  artificially synthesized primer sequence
aataataatagcggccgcttttttttttttttttttttaagtcactcttttctctgc

SEQ ID NO: 16 - DNA, 45 bp, Artificial Sequence
  artificially synthesized primer sequence
tttttgcggccgcgctgctggtcattattcgtcgtccattggtgt

SEQ ID NO: 17 - DNA, 45 bp, Artificial Sequence
  artificially synthesized primer sequence
aaaaaaaagatctggagtcttcttcggtaacgactttgccctttg

SEQ ID NO: 18 - DNA, 38 bp, Artificial Sequence
  artificially synthesized primer sequence
aaaaaaaaaagcggccgccccctacagagttttgcaag

SEQ ID NO: 19 - DNA, 39 bp, Artificial Sequence
  artificially synthesized primer sequence
aaaaaaagatcttggagtctaatattgaataccataccg

SEQ ID NO: 20 - DNA, 30 bp, Artificial Sequence
  artificially synthesized primer sequence
cctaacctaacctaacctaacctaacctaa

SEQ ID NO: 21 - DNA, 30 bp, Artificial Sequence
  artificially synthesized primer sequence
ttaggttaggttaggttaggttaggttagg

SEQ ID NO: 22 - DNA, 65 bp, Artificial Sequence
  telomeric repeat sequence
ttaggttaggttaggttaggttaggttaggttaggttaggttaggttaggttaggttaggttagg

SEQ ID NO: 23 - DNA, 65 bp, Artificial Sequence
  telomeric repeat sequence
cctaacctaacctaacctaacctaacctaacctaacctaacctaacctaacctaacctaacctaa

SEQ ID NO: 24 - DNA, 10 bp, Artificial Sequence
  3′ boundary sequence of an artificially constructed SART1 delivertive
catcgatacc

SEQ ID NO: 25 - DNA, 10 bp, Artificial Sequence
  3′ boundary sequence of an artificially constructed SART1 delivertive
tcgttgggtt

SEQ ID NO: 26 - DNA, 10 bp, Artificial Sequence
  5′ boundary sequence of an artificially constructed SART1 delivertive
aagtatttta

SEQ ID NO: 27 - DNA, 10 bp, Artificial Sequence
  5′ boundary sequence of an artificially constructed SART1 delivertive
tggcgaaaca

SEQ ID NO: 28 - DNA, 10 bp, Artificial Sequence
  5′ boundary sequence of an artificially constructed SART1 delivertive
acgttatata

SEQ ID NO: 29 - DNA, 10 bp, Artificial Sequence
  5′ boundary sequence of an artificially constructed SART1 delivertive
atattagata

SEQ ID NO: 30 - DNA, 10 bp, Artificial Sequence
  5′ boundary sequence of an artificially constructed SART1 delivertive
ttgttttata

SEQ ID NO: 31 - DNA, 10 bp, Artificial Sequence
  5′ boundary sequence of an artificially constructed SART1 delivertive
tttatacatg

SEQ ID NO: 32 - DNA, 10 bp, Artificial Sequence
  5′ boundary sequence of an artificially constructed SART1 delivertive
acccaatgtg

SEQ ID NO: 33 - DNA, 10 bp, Artificial Sequence
  5′ boundary sequence of an artificially constructed SART1 delivertive
aagtatttta

SEQ ID NO: 34 - DNA, 10 bp, Artificial Sequence
  5′ boundary sequence of an artificially constructed SART1 delivertive
aagtatttta

SEQ ID NO: 35 - DNA, 11 bp, Artificial Sequence
  5′ boundary sequence of an artificially constructed SART1 delivertive
gaagtatttta

SEQ ID NO: 36 - DNA, 11 bp, Artificial Sequence
  5′ boundary sequence of an artificially constructed SART1 delivertive
caagtatttta

SEQ ID NO: 37 - DNA, 12 bp, Artificial Sequence
  5′ boundary sequence of an artificially constructed SART1 delivertive
tcaagtatttta

SEQ ID NO: 38 - DNA, 10 bp, Artificial Sequence
  5′ boundary sequence of an artificially constructed SART1 delivertive
aagtatttta

SEQ ID NO: 39 - protein, 12 aa, Artificial Sequence
  N-terminal sequence of APE domain of SART1/TRAS1 chimeric retrotransposon
MTSSAAAAPYRV

SEQ ID NO: 40 - protein, 12 aa, Artificial Sequence
  C-terminal sequence of APE domain of SART1/TRAS1 chimeric retrotransposon
IRLQDLESSSVT

SEQ ID NO: 41 - DNA, 10 bp, Artificial Sequence
  3′ boundary sequence of SART1
catcgatacc

SEQ ID NO: 42 - DNA, 10 bp, Artificial Sequence
  3′ boundary sequence of TRAS1
agagtgactt

SEQ ID NO: 43 - DNA, 10 bp, Artificial Sequence
  3′ boundary sequence of SART1/TRAS1 chimeric retrotransposon
catcgatacc

SEQ ID NO: 44 - DNA, 10 bp, Artificial Sequence
  3′ boundary sequence of an artificially constructed SART1 delivertive
tggtggtgag

SEQ ID NO: 45 - DNA, 10 bp, Artificial Sequence
  3′ boundary sequence of an artificially constructed SART1 delivertive
ggtggtgagg

SEQ ID NO: 46 - DNA, 10 bp, Artificial Sequence
  3′ boundary sequence of an artificially constructed SART1 delivertive
agggtatagg

SEQ ID NO: 47 - DNA, 10 bp, Artificial Sequence
  3′ boundary sequence of an artificially constructed SART1 delivertive
gtatagggcg

SEQ ID NO: 48 - DNA, 10 bp, Artificial Sequence
  3′ boundary sequence of an artificially constructed SART1 delivertive
ggagctcgtt

SEQ ID NO: 49 - DNA, 10 bp, Artificial Sequence
  3′ boundary sequence of an artificially constructed SART1 delivertive
tgggcgggtt

SEQ ID NO: 50 - DNA, 10 bp, Artificial Sequence
  3′ boundary sequence of an artificially constructed SART1 delivertive
tcgttgggtt

SEQ ID NO: 51 - protein, 189 aa, Artificial Sequence
  consensus sequence of Exo_endo_phos domain
LKVLTWNVNGLRALLLLELLREDPDVLGLQEVKLSELLLLLLGYYGFGGGGGKGGVAILSKLPLLSVILGIDLIRVISTSGTFVVVNTHLPAGDERLAQLAELLDFLSFKSDPVILLGDFNARPDEWDSLLEIGKIGFPPTYWSYRGSSEKKRTPSRLDRILVSGLLRVVSLILLEVLGSDHRPVLATL

SEQ ID NO: 52 - DNA, 461 bp, Artificial Sequence
  3′ UTR of SART1
ggaccgtcgggcgtcgggcgcgggggcgcctgatgctcgacacgatagtccagtggtggtggtgagggtatagggcgctgtggctccggtgcctacctcacgaagaagttgcggtcagcaatggccgacgttgatcccgccgttcgtcaggctgggagccggtgtggggggcctgcggggcgcgtttccctgtggtatcgtaggttccccctatgccggaaagataggagctcgttgggttttagtcggtagtcgttaagctgggcgggttcggcgcgagctgaactcagcccagcgcgcctttttcaaggcgtagtctccgtggactaattggtcgagggcgcggacctcggttcgcgacttcttcctgtttcttccaccggaggcgcggagtccgacataacccggtccgacccccgtcggccgggtatccgtaaagactgggattccccatcgatacc

SEQ ID NO: 53 - DNA, 27 bp, Artificial Sequence
  artificially synthesized primer sequence
ttttttgaattcggaccgtcgggcgtc

SEQ ID NO: 54 - DNA, 45 bp, Artificial Sequence
  artificially synthesized primer sequence
ttttttggatccagatcttttttttttttttttttttggtatcga